(19) United States
(12) Patent Application Publication
(10) Pub. No.: US 2024/0012078 A1
(43) Pub. Date: Jan. 11, 2024

(54) MRI IN AN INHOMOGENEOUS FIELD WITH NO PULSED GRADIENTS

(71) Applicant: Ramot at Tel-Aviv University Ltd., Tel-Aviv (IL)

(72) Inventors: Uri NEVO, Tel-Aviv (IL); David CORCOS, Tel-Aviv (IL)

(73) Assignee: Ramot at Tel-Aviv University Ltd., Tel-Aviv (IL)

(21) Appl. No.: 18/021,577

(22) PCT Filed: Aug. 17, 2021
(86) PCT No.: PCT/IL2021/051003

§ 371 Date: Feb. 16, 2023

Related U.S. Application Data
(60) Provisional application No. 63/066,356, filed on Aug. 17, 2020.

(51) Int. Cl. G01R 33/56; A61B 5/055; A61B 5/00; G06T 7/00

(52) U.S. Cl. CPC G01R 33/5608; A61B 5/055; A61B 5/0042; G06T 7/0012; G06T 2207/10088; G06T 2207/30016; A61B 2576/026

(57) ABSTRACT

A method of scanning an object in a FOV by acquiring an MRI signal from the object at different projections of a spatially encoding magnetic field, using a plurality of receiving antennas, and reconstructing an MRI image of the object, comprising:
a) at each projection, acquiring the MRI signal from the receiving antennas;
b) filtering the received signal, for at least some of the projections, by applying different time windows to different components of the signal in different frequency bands, and/or received by different receiver antennas, resulting in a filtered received signal vector whose components describe the filtered received signal as a function of time, at each projection, for one or more receiver antennas; and
c) reconstructing an image as a vector whose components describe a weighted or unweighted net magnetization at each voxel in the FOV, that would be expected to produce the filtered received signal vector.

### RELATED APPLICATION/S

This application claims the benefit of priority of U.S. Provisional Patent Application No. 63/066,356 filed on Aug. 17, 2020, the contents of which are incorporated herein by reference in their entirety.

### FIELD AND BACKGROUND OF THE INVENTION

The present invention, in some embodiments thereof, relates to a magnetic resonance imaging (MRI) device and image reconstruction method, using a highly inhomogeneous magnetic field and, more particularly, but not exclusively, to such a device and method using an open magnet configuration, and/or not using pulsed gradient coils.

U.S. Pat. No. 9,910,115 to Wald et al describes a portable magnetic resonance imaging (“MRI”) system that uses static magnetic field inhomogeneities in the main magnet for encoding the spatial location of nuclear spins. Also provided is a spatial-encoding scheme for a low-field, low-power consumption, light-weight, and easily transportable MRI system. In general, the portable MRI system spatially encodes images using spatial inhomogeneities in the polarizing magnetic field rather than using gradient fields. Thus, an inhomogeneous static field is used to polarize, readout, and encode an image of the object. To provide spatial encoding, the magnet is rotated around the object to generate a number of differently encoded measurements. An image is then reconstructed by solving for the object most consistent with the data.

Klaas P. Pruessmann, Markus Weiger, Markus B. Scheidegger, and Peter Boesiger, “SENSE: Sensitivity encoding for fast mri,” Magnetic Resonance in Medicine, 42(5):952-962, 1999, presents new theoretical and practical concepts for considerably enhancing the performance of magnetic resonance imaging (MRI) by means of arrays of multiple receiver coils. Sensitivity encoding (SENSE) is based on the fact that receiver sensitivity generally has an encoding effect complementary to Fourier preparation by linear field gradients. Thus, by using multiple receiver coils in parallel scan time in Fourier imaging can be considerably reduced. The problem of image reconstruction from sensitivity encoded data is formulated in a general fashion and solved for arbitrary coil configurations and k-space sampling patterns. Special attention is given to the currently most practical case, namely, sampling a common Cartesian grid with reduced density. For this case the feasibility of the proposed methods was verified both in vitro and in vivo. Scan time was reduced to one-half using a two-coil array in brain imaging. With an array of five coils double-oblique heart images were obtained in one-third of conventional scan time.

Additional background art includes Cooley et al, “Design of Sparse Halbach Magnet Arrays for Portable MRI Using a Genetic Algorithm,” IEEE TRANSACTIONS ON MAGNETICS, VOL. 54, NO. 1, JANUARY 2018; Weber et al, “Local Field of View Imaging for Alias-Free Undersampling with Nonlinear Spatial Encoding Magnetic Fields,” Magnetic Resonance in Medicine 71:1002-1014 (2014); Sarty et al, “Magnetic resonance imaging with RF encoding on curved natural slices,” Magnetic Resonance Imaging 46 (2018) 47-55; Schultz et al, “MR Image Reconstruction from Generalized Projections,” Magnetic Resonance in Medicine 72:546-557 (2014); Schultz et al, “Radial Imaging With Multipolar Magnetic Encoding Fields,” IEEE TRANSACTIONS ON MEDICAL IMAGING, VOL. 30, NO. 12, DECEMBER 2011; Schultz et al, “Reconstruction of MRI Data Encoded With Arbitrarily Shaped, Curvilinear, Nonbijective Magnetic Fields,” Magnetic Resonance in Medicine 64:1390-1404 (2010); Stockmann et al, “Transmit Array Spatial Encoding (TRASE) using broadband WURST pulses for RF spatial encoding in inhomogeneous B0 fields,” Journal of Magnetic Resonance 268 (2016) 36-48; Cooley et al, “Two-Dimensional Imaging in a Lightweight Portable MRI Scanner without Gradient Coils,” Magnetic Resonance in Medicine 73:872-883 (2015); Xiaobo Qu et al, “COMPRESSED SENSING MRI WITH COMBINED SPARSIFYING TRANSFORMS AND SMOOTHED [symbol] ₀ NORM MINIMIZATION,” ICASSP 2010, p.626-629; Candès, “Compressive sampling,” Proceedings of the International Congress of Mathematicians, Madrid, Spain, 2006; Y. C. Eldar, Sampling Theory: Beyond Bandlimited Systems, Chapter 11, “Generalized Sampling Methods 049033, Compressed Sensing”; Donoho, “Compressed Sensing,” IEEE TRANSACTIONS ON INFORMATION THEORY, VOL. 52, NO. 4, APRIL 2006; Lustig et al, “Sparse MRI: The Application of Compressed Sensing for Rapid MR Imaging,” Magnetic Resonance in Medicine 58:1182-1195 (2007); Venkatakrishnan et al, “Plug-and-Play Priors for Model Based Reconstruction,” May 29, 2013, ECE Technical Reports, Purdue e-Pubs, Purdue University; McDaniel et al, “The MR Cap: A single-sided MRI system designed for potential point-of-care limited field-of-view brain imaging,” Magn Reson Med. 2019;00:1-15; Beck et al, “A Fast Iterative Shrinkage-Thresholding Algorithm for Linear Inverse Problems,” SIAM J. IMAGING SCIENCES Vol. 2, No. 1, pp. 183-202 (2009); and Sharp et al, “MRI Using Radiofrequency Magnetic Field Phase Gradients,” Magnetic Resonance in Medicine 63:151-161 (2010).

### SUMMARY OF THE INVENTION

An aspect of some embodiments of the invention concerns an MRI device and method of magnetic resonance image reconstruction using an adaptive acquisition time, in which the received MRI signal is filtered so that different components have different acquisition times, including different frequency components and components from different receiver antennas.

There is thus provided, according to an exemplary embodiment of the invention, a method of scanning an object in field of view (FOV) by acquiring an MRI signal from the object in the FOV at a plurality of different projections of a spatially encoding magnetic field, using a plurality of receiving antennas, and reconstructing an MRI image of the object in the FOV comprising a plurality of voxels, the method comprising:
a) at each of the projections, acquiring the MRI signal from the receiving antennas, and recording data of the signal;
b) filtering the received signal with a signal filter, for at least some of the projections, by applying different time windows to at least some different components of the signal in different frequency bands, or received by different receiver antennas, or both, resulting in a filtered received signal vector whose components describe the filtered received signal as a function of time, at each projection, for one or more receiver antennas; and
c) reconstructing an image as a vector whose components describe a weighted or unweighted net magnetization at each voxel in the FOV, that would be expected to produce the filtered received signal vector.

Optionally, recording the data comprises recording the data separately for at least some different receiver antennas or sets of receiver antennas, and filtering the received signal results in a filtered received signal vector whose components describe the filtered received signal as a function of time, at each projection, for each receiver antenna or set of receiver antennas for which the data is recorded separately.

Optionally, at least some of the components of the signal received from different receiver antennas at a same or overlapping frequency band have different time windows.

Optionally, the components of the filtered received signal vector for each projection, for each receiver antenna or set of receiver antennas for which the data is recorded separately, comprise values of the signal in different time intervals.

Optionally, the components of the filtered received signal vector for each projection, for each receiver antenna or set of receiver antennas for which the data is recorded separately, comprise values of the signal in different frequency bands.

Optionally, finding the reconstructed image vector comprises finding an image vector that minimizes a positive definite measure of a difference between the filtered received signal vector and a filtered signal vector that would be expected for the image vector.

Optionally, the expected filtered signal vector for an image vector is an encoding matrix operating on the image vector, and finding the reconstructed image vector comprises multiplying the filtered received signal by an inverse or pseudo-inverse of the encoding matrix to find an image vector that would be expected to produce the filtered received signal vector.

Optionally, finding the reconstructed image vector comprises finding an image vector that minimizes a sum of:
a) a positive definite measure of a difference between the filtered received signal vector and a filtered signal vector that would be expected for the image vector; and
b) a regularization term that depends on the image vector.

Optionally, the positive definite measure of a difference between the filtered received signal vector and the expected filtered signal vector is a sum or weighted sum of squares of vector components of the difference.

Optionally, the regularization term comprises a Tikhonov regularization term.

Alternatively or additionally, the regularization term comprises a constant multiplying a sum or weighted sum of absolute values of vector components of the image vector transformed by a matrix.

Optionally, the matrix is a sparsifying transform.

Optionally, the sparsifying transform is a wavelet transform.

In an exemplary embodiment of the invention, the spatially encoding magnetic field is produced by a magnetic field source that rotates rigidly around at least one axis around the FOV, the different projections comprise a plurality of different rotation angles of the magnetic field source around the axis relative to the FOV, and the number of different rotation angles around the axis is lower by at least a factor of 2 compared to π/2 times a width of the FOV divided by a distance Δx that is resolved in the reconstructed image, times a ratio of an average magnetic field gradient in the FOV to an effective minimum magnetic field gradient in the FOV.

Alternatively, the spatially encoding magnetic field is produced by a magnetic field source comprising a plurality of elements at least some of which move and/or rotate relative to each other to produce the different projections, such that for at least two of the projections, the spatially encoding magnetic field for one of the projections is a non-rigid distortion of the spatially encoding magnetic field for the other projection.

Optionally, the elements comprise one or more fixed elements that do not change their position or orientation between different projections, and one or more moving elements that do change their position or orientation between different projections.

Optionally, the FOV includes at least part of a human subject's brain inside the subject's cranium, and for at least one projection the elements are located outside the subject's head, in a configuration with at least some of the elements sufficiently close to the subject's head that there is not room for the magnetic field source to rotate rigidly 180 degrees around the head.

Optionally, finding the reconstructed image vector comprises searching for the image vector that minimizes the sum.

Alternatively, finding the reconstructed image vector comprises searching for the image vector that minimizes the positive definite measure of the difference.

Optionally, searching for the image vector comprises using an iterative search method.

Alternatively, finding the image vector comprises calculating the image vector from an analytic expression for the image vector in terms of the filtered received signal vector.

Optionally, values of the components of the filtered signal vector that would be expected for a given weighted or unweighted net magnetization as a function of voxel in the FOV, for each projection, are values that would be expected if, for every pair of different projections, the spatially encoding magnetic fields are rigid rotations of each other, relative to the FOV, over some angle around some axis.

Optionally, said values are values that would be expected if, for all projections, the spatially encoding magnetic fields are rigid rotations of each other, relative to the FOV, over some angle around a single axis.

In an exemplary embodiment of the invention, for those projections for which the received signal is filtered, the time window has a standard deviation that is narrower, on average, for frequency component and receiver antenna combinations that have a characteristic magnetic field gradient magnitude that is higher than an average magnetic field gradient magnitude in the FOV, than for frequency component and receiver antenna combinations that have a characteristic magnetic field gradient magnitude that is lower than the average magnetic field gradient magnitude in the FOV.

Optionally, the standard deviations of the time windows of the frequency component and receiver antenna combinations are linearly correlated with an inverse of the characteristic magnetic field gradient magnitudes of the frequency component and receiver antenna combinations, with a correlation greater than 0.5.

Optionally, the voxels of the FOV have width of about a same value Δx, and a best linear fit of the standard deviation of the time window to the inverse of the characteristic magnetic field gradient magnitude Gₗ has the standard deviation of the time window between 1/(γGₗΔx) and 2.5/(γGₗΔx), where γ is the gyromagnetic ratio.

Optionally, the MRI signal is received substantially only from a FOV only one voxel thick, with the voxels arranged in two dimensions in the FOV, and wherein, for each projection in at least one set of the projections that together contribute most of the MRI signal's energy, for a set of the voxels from which at least 90% of the MRI signal's energy is received, the spatially encoding magnetic field better distinguishes between components of the MRI signal received from each of those voxels and at least one adjacent voxel, than it distinguishes between components of the MRI signal received from different locations within the voxel.

Optionally, the spatially encoding magnetic field is produced by a magnetic field source that rigidly rotates around the FOV on only one axis, and the different projections comprise different rotation angles of the magnetic field source around the axis with respect to the FOV.

Optionally, the FOV comprises a volume with voxels arranged in three dimensions.

Optionally, the spatially encoding magnetic field is produced by a magnetic field source comprising one or more elements at least some of which change their positions or orientations or both, for the different projections, and wherein the different projections correspond to different values of two independent degrees of freedom, each degree of freedom being a different combination of a position, an orientation, or both, of one or more of the elements.

Optionally, the spatially encoding magnetic field is produced by a magnetic field source that rotates rigidly around the FOV on two or more axes, and the different projections comprise different rotation angles of the magnetic field source around the two or more axes with respect to the FOV.

Alternatively, the spatial encoding magnetic field is produced by a magnetic field source that rotates rigidly around the FOV on one axis and translates along the axis, and the different projections comprise different combinations of rotation angle and translation distance of the magnetic field source, with respect to the FOV.

Alternatively, the method also comprises, for each of the projections of the spatially encoding magnetic field:
a) exciting nuclear spins in the FOV with an MRI excitation pulse; and
b) receiving the MRI signal from the excited nuclear spins over a time interval after the excitation pulse;
   wherein the spatial encoding magnetic field is produced by a magnetic field source that rotates rigidly around the FOV on one axis, the different projections comprise different rotation angles of the magnetic field source around the axis with respect to the FOV, and components of the signal coming from different distances along the axis are distinguished by the excitation pulse at each projection having a phase that depends nonlinearly on distance along the axis.

Optionally, filtering the received signal comprises using software to calculate the components of the filtered received signal vector from stored data of a received unfiltered signal.

Alternatively, filtering the received signal comprises receiving different frequency components of the received signal using different receivers, and recording data of each frequency component only during the time window for that frequency component.

Optionally, for at least one projection, the spatial encoding magnetic field has field gradient magnitudes at different locations within the FOV that differ by at least a factor of 1.5.

Optionally, the standard deviations of the time windows for two or more different frequency component and receiver antenna combinations of the filtered signal, that together contribute at least 10% to a total energy of the filtered signal for that projection, have a standard deviation that is at least 20% of their mean value.

Optionally, the spatially encoding magnetic field has a maximum value in the FOV that is 0.5 tesla or less.

Optionally, the spatially encoding magnetic field has a maximum value in the FOV that is 0.3 tesla or less.

There is further provided, in accordance with an exemplary embodiment of the invention, a magnetic resonance imaging device for reconstructing an image of a FOV, comprising:
a) a magnetic field source that produces a spatially encoding magnetic field in the FOV, that has a different dependence on position in the FOV at each of a plurality of different projections;
b) one or more MRI excitation antennas that produce an electromagnetic field that resonantly excites nuclear spins in the FOV;
c) a plurality of MRI receiver antennas that receive MRI signals from the excited nuclear spins in the FOV, and one or more MRI receivers that record data of the received MRI signals separately for each projection for at least some different receiver antennas or sets of receiver antennas;
d) a signal filtering module that, for at least some of the projections, filters the received signal by applying time windows of different widths to at least some different components of the signal in different frequency bands, from different receiver antennas, or both, resulting in a filtered received signal vector whose components describe the filtered received signal as a function of time or frequency, at each projection, for each receiver antenna or set of receiver antennas for which the data is recorded separately; and
e) an image reconstruction module configured to find a reconstructed image vector whose components describe a weighted or unweighted net magnetization at each voxel in the FOV, that would be expected to produce the filtered received signal vector.

Optionally, the one or more MRI receivers record data of the unfiltered received MRI signal, and the signal filtering module is configured to calculate the filtered received signal vector from the data of the unfiltered received MRI signal.

Optionally, at least some of the one or more MRI receivers are configured to separately record data of the received MRI signal at each of a plurality of frequency components, and the signal filtering module is configured to limit the recording of data for each frequency component to the time window for that frequency component.

Optionally, the device comprises a magnetic field source rotator configured to rigidly rotate the magnet field source to a plurality of different orientations around one or more axes relative to the FOV, maintaining a same configuration of the spatially encoding magnetic field relative to the magnetic field source for each of the orientations, the different projections corresponding to the different orientations of the magnetic field source.

Optionally, the magnetic field source comprises an open magnet.

Optionally, the magnetic field source comprises a plurality of magnets, and at least some of the receiver antennas are each located much closer to a different one of the magnets than to the other magnets.

Optionally, the magnetic field source comprises one or more permanent magnets.

Optionally, the one or more permanent magnets comprise a cap-shaped magnet configuration.

Optionally, the magnetic field source comprises at least two elements, the device also comprising a field source element motion system configured to move or rotate one of the elements relative to the other element such that the motion or rotation distorts the spatially encoding magnetic field produced by the magnetic field source.

There is further provided, in accordance with an exemplary embodiment of the invention, a method of scanning an object in a field of view (FOV) by acquiring an MRI signal from the object in the FOV at a plurality of different projections of a spatially encoding magnetic field generated by a magnetic field source comprising a plurality of magnetic elements at least some of them moveable, and reconstructing an MRI image of the object in the FOV, the method comprising:
a) at each of the projections, acquiring the MRI signal from the FOV;
b) after at least some of the projections, using the magnetic field source to change the spatially encoding magnetic field for the next projection, including, for at least one of those projections, moving at least one of the magnetic elements relative to another of the magnetic elements such that the spatially encoding magnetic field of the next projection is a distortion of the spatially encoding magnetic field of the at least one projection; and
c) reconstructing the MRI image of the object in the FOV from the acquired MRI signal.

Optionally, reconstructing the MRI image of the object in the FOV comprises using an encoding matrix that transforms a given image vector of the FOV to a filtered or unfiltered MRI signal vector that the image vector would be expected to produce, and the method also comprises, before acquiring the MRI signal for any of the projections, calibrating the spatially encoding magnetic field at each projection by:
a) placing in the FOV a phantom that produces a known image;
b) acquiring an MRI signal from the FOV for each of the projections;
c) reconstructing an image of the phantom from the MRI signal acquired at each projection;
d) comparing the reconstructed image of the phantom with the known image of the phantom;
e) adjusting one or more components of the encoding matrix, based on differences between the reconstructed image of the phantom and the known image of the phantom, to values that would make the reconstructed image of the phantom more closely match the known image of the phantom; and
f) replacing the phantom in the FOV with the object to be scanned.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 schematically shows a system for producing magnetic resonance images using an inhomogeneous magnetic field produced by one or more permanent magnets that move around to generate different projections of a spatially encoding magnetic field, according to an exemplary embodiment of the invention;

FIG. 2 is a block diagram showing different software modules used by the computer shown in the system of FIG. 1, including a signal filtering module that filters the received MRI signal by using different acquisition time windows for different components of the signal that come from locations with different magnetic field gradients, and an image reconstruction module that reconstructs an image from the filtered signal, according to an exemplary embodiment of the invention;

FIG. 3 schematically shows permanent magnets and receiver coils for a system for producing magnetic resonance images using an inhomogeneous magnetic field produced by the permanent magnets instead of pulsed gradient coils, with an alternative configuration to the configuration shown in FIG. 1, showing the positions of the magnets in two different projections of the spatially encoding magnetic field in which the field is distorted between one projection and the other, according to an exemplary embodiment of the invention;

FIG. 4 schematically shows a configuration of permanent magnets for a system that produces magnetic resonance images using an inhomogeneous magnetic field produced by the permanent magnets instead of pulsed gradient coils, with an alternative configuration to FIGS. 1 and 3, in which the magnets rotate rigidly around an axis so that the spatially encoding magnetic field is not distorted between different projections, according to an exemplary embodiment of the invention;

FIG. 5 is a flow chart for a method of producing magnetic resonance images using the system shown in FIGS. 1 and 2, or with alternative magnet configurations such as those shown in FIGS. 3 and 4, according to an exemplary embodiment of the invention;

FIG. 6 is a flow chart showing details of how the image is optionally reconstructed in the method of FIG. 5, according to an exemplary embodiment of the invention;

FIG. 7 schematically shows the magnetization as a function of position in a 1-D field of view with two narrow objects at locations with different magnetic field gradients, to illustrate how the method of FIGS. 5 and 6 can be used to reconstruct an image according to an exemplary embodiment of the invention;

FIG. 8 schematically shows MRI signals in the time domain from the two objects shown in FIG. 7, using a relatively long acquisition for both objects time that produces aliasing artifacts for the object located at the higher field gradient;

FIG. 9 schematically shows a reconstructed image of the two objects of FIG. 7, including the aliasing artifacts, using the MRI signals shown in FIG. 8;

FIG. 10 schematically shows MRI signals in the time domain from the two objects shown in FIG. 7, using a truncated acquisition time for both objects that avoids aliasing artifacts, but produces a loss of resolution for the object located at the lower field gradient;

FIG. 11 schematically shows a reconstructed image of the two objects of FIG. 7, including the loss of resolution, using the MRI signals shown in FIG. 10;

FIG. 12 schematically shows a contour plot of the magnetic field in the 2-D field of view for the magnet configuration shown in FIG. 4;

FIG. 13 schematically shows an alternative magnet configuration to the configuration shown in FIG. 4, in the form of a magnetic cap, in which the magnet rotates rigidly so that the magnetic field does not distort in going between different projections; and

FIG. 14 schematically shows a contour plot of the magnetic field in the 2-D field of view for the magnet configuration shown in FIG. 13.

### DESCRIPTION OF SPECIFIC EMBODIMENTS OF THE INVENTION

The present invention, in some embodiments thereof, relates to a magnetic resonance imaging (MRI) device and image reconstruction method, using a highly inhomogeneous magnetic field and, more particularly, but not exclusively, to such a device and method using an open magnet configuration, and/or not using pulsed gradient coils.

An aspect of some embodiments of the invention concerns a method of reconstructing a magnetic resonance image of a field of view (FOV) with a spatially encoding magnetic field, from a received MRI signal from the FOV, where the received signal is filtered using an adaptive acquisition time. The acquisition time is longer for at least some components of the signal that come from locations of the FOV with lower magnetic field gradient, and shorter for at least some components of the signal that come from locations of the FOV with higher magnetic field gradient. This adaptive acquisition time potentially allows the reconstructed image to have a desired resolution, for example uniform resolution over the FOV or higher resolution in certain parts of the FOV, even across regions that have very different field gradients, with high resolution even in regions with low field gradient, while avoiding or reducing aliasing image artifacts even in regions of high field gradient. The use of adaptive acquisition time may make it possible to obtain the advantages of an open magnet MRI system without gradient coils, such as being much less expensive and more portable than a conventional MRI system, while having a better image quality than existing open magnet MRI systems.

For example, if the FOV is divided into voxels of width Δx, which may be constant across the FOV or may vary with location, the full image resolution is obtained, without aliasing artifacts, when the acquisition time is just long enough so that two nuclear spins, separated by Δx in the direction of a field gradient of magnitude G, will get out of phase in their precession by π radians, so the acquisition time is π/(γGΔx), where γ is the gyromagnetic ratio of the nuclear spins. If the nuclear spins get out of phase by less than π radians in an acquisition time, in a location of the FOV, then the image resolution will tend to be lower in that location. If the nuclear spins get out of phase by more than π radians in an acquisition time, in a location of the FOV, then the image will tend to have aliasing artifacts in that location. If the magnitude of GΔx is very different at different locations in the FOV, then a better image quality is potentially obtained by using a longer acquisition time at locations with smaller GΔx, for example with smaller field gradient G, and a shorter acquisition time at locations with larger GΔx, for example with larger field gradient G.

It should be understood that although the term “voxel” often refers to an element of a 3-D image, and “pixel” often refers to an element of a 2-D image, the terms “voxel” and “pixel” are sometimes used interchangeably herein. It should not be assumed that use of “voxel” implies that only a 3-D image is meant, or that use of “pixel” implies that only a 2-D image is meant. When only a 2-D image is meant, or only a 3-D image is meant, this will be stated explicitly.

Optionally, adaptive acquisition time is implemented by having different acquisition times for at least some different components of the signal, including different frequency components, and components of the signal received by different receiver antennas, which are recorded separately. The signal coming from a given location in the FOV has a frequency centered at the resonance frequency of the nuclear spins at that location, which depends on the magnitude of the magnetic field there. For example, consider the received signal as divided into a large number of narrow frequency bands. The signal from a given frequency band originates in a narrow slice, often a curved slice, of the FOV, where the magnitude of the spatially encoding magnetic field in the FOV has a narrow range of values, such that the precession frequencies of nuclear spins in that slice are within that frequency band. Then, in this implementation, the acquisition time chosen for that frequency component depends on a characteristic field gradient magnitude for locations in that slice, with the acquisition time being longer for frequency bands for which the characteristic field gradient for the corresponding slice is lower, and shorter for frequency bands for which the characteristic field gradient for the corresponding slice is greater. For example, the acquisition time is optionally longer for most frequency bands for which the characteristic field gradient is greater than the average magnitude of field gradient over the FOV, than for most frequency bands for which the characteristic field gradient is smaller than the average magnitude of field gradient over the FOV.

The characteristic field gradient for a given frequency band is optionally defined, for example, as a mean value of the magnitude of the field gradient in the region of the FOV where the precession frequency of the nuclear spins is within that frequency band. Optionally, this mean value is weighted toward or limited to parts of the FOV that are of greater interest, for example optionally greater weight is given to locations closer to the center of the FOV. Optionally, for signal components that come from different receiver antennas, more weight is given to locations for which the sensitivity is greater for that receiver antenna, or for which the sensitivity is greater relative to other receiver antennas. For example, the weight given to a location is proportional to the sensitivity of that receiver antenna at that location, optionally normalized to the sum of the sensitivities of all the antennas at that location. Optionally, the characteristic field gradient is a different function of frequency, for different receiver antennas, and for at least one frequency band, two different receiver antennas will have different characteristic field gradients. If the ranges of the frequency bands are defined differently for the two different receiver antennas, then there will be at least one pair of overlapping frequency bands, for the two receiver antennas, for which the characteristic field gradient is different. Alternatively, the characteristic field gradient is the same function of frequency for different receiver antennas, in spite of their having different sensitivity as a function of location, and the different sensitivity as a function of position is not taken into account in defining the characteristic field gradient for different receiver antennas.

This implementation of adaptive acquisition time may be especially useful if the spatially encoding magnetic field is substantially nonlinear, with a substantial range of magnitudes of field gradient across the FOV, for example, with field gradient magnitudes that vary by a factor between 1.2 and 1.5, or between 1.5 and 2, or between 2 and 3, or between 3 and 5, or at least a factor of 5, across the FOV. This is likely to be true, for example, in an MRI device that uses an open magnet configuration to produce the spatially encoding magnetic field, and/or uses permanent magnets to produce the spatially encoding magnetic field, as opposed to conventional MRI devices with a FOV inside a bore of an electromagnet with very uniform field, and with spatial encoding using gradient coils that produce a very linear gradient across the FOV. In a highly nonlinear spatially encoding magnetic field, the range of field gradient magnitude over a slice with a given field magnitude is likely to be much smaller than the total range of field gradient magnitude over the FOV, since higher field gradients will generally occur in regions with higher field, and lower field gradients will generally occur in regions with lower field. Then the acquisition time, for each frequency component of the signal, can be well adapted to the magnitude of the field gradient at the locations where that frequency component is coming from, potentially allowing the reconstructed image to have uniformly good resolution and a low level of aliasing image artifacts. Having the acquisition time also depend on the receiver antenna can make it even better adapted to the magnitude of the field gradient at the locations where the signal component is coming from, since different antennas are more sensitive to different locations in the FOV.

MRI devices using open magnetic field configurations, and using permanent magnets rather than coils to produce their spatially encoding magnetic field, are potentially much less expensive, and much more portable, than conventional MRI devices, but the large range in magnetic field gradients over the FOV often results in poorer quality of reconstructed images, with lower resolution in regions where the field gradient is lower, and/or aliasing image artifacts in regions where the field gradient is greater. The use of adaptive acquisition time can potentially allow such inexpensive and portable MRI devices to produce images of much better quality than they could otherwise produce, possibly as good quality as a much more expensive MRI device, as described for example in <www(dot)en(dot)Wikipedia(dot)org/wiki/Physics_of_magnetic_resonance_imaging> and references therein.

To reconstruct an image from MRI signals, signals are received from the FOV using a plurality of different projections of the spatially encoding magnetic field. In conventional MRI, the different projections of the magnetic field are produced by gradient coils with controllable currents, that successively produce field gradients of different magnitude and direction in the FOV. Alternatively, different projections of the magnetic field can be produced without using gradient coils of controllable current, or even using only permanent magnets. This can be done, for example, by rotating a magnetic field source around a FOV, optionally rigidly rotating the magnetic field source so that the magnetic field has the same configuration relative to the magnetic field source, and receiving MRI signals from the FOV at each of a plurality of different rotation angles, successively.

Optionally, the received signal at each projection of the spatially encoding magnetic field is expressed as a vector, with each component of the vector being a value of the signal at a given time interval, within the acquisition time over which the signal is acquired, generally a complex value representing both the amplitude and phase of the signal. Alternatively, each component of the vector is a value of the received signal in a given frequency band, or a value of the signal in some other set of basis functions, such as cubic splines, or wavelets. In all of these cases, the set of components of the vector can be used to find the value (amplitude and phase) of the signal as a discretized function of time, and the components of the vector, for that projection of the magnetic field, are said to describe the signal as a function of time, for that projection. Optionally, the signal vectors for different projections are stacked together to form a signal vector for the entire received signal, the components of which describe the signal as a function of time for each projection.

Optionally, if there is more than one receiver, for example receivers that have different sensitivity to signals emitted from different spatial locations, or at different frequencies, then the received signal from each receiver is recorded separately, assigned to its own vector, and the vectors from the different receivers for each of the different projections are stacked together, to make the signal vector for the entire received signal. For example, there are 2 to 5 different receivers, or 6 to 10 different receivers, or 11 to 20 different receivers, or more than 20 different receivers. This has the potential advantage that the differences in received signal at each receiver can be used to obtain more accurate information about the image. Alternatively, signals from different receivers are simply added together, for each projection, and only the sum is recorded in the signal vector. This has the potential advantage that the signal vector will have fewer components, and it may be easier to calculate the reconstructed image.

Optionally, the received signal is filtered by limiting each frequency component, for each projection, to its own acquisition time, which in general may be less than the total time over which the signal is acquired for that projection, and which in general may be different for different frequency components and for different receiver antennas. This filtering of the signal is how the adaptive acquisition time, described above, is optionally implemented. Optionally, values of the unfiltered signal are recorded, for each projection, over the total acquisition time for that projection, and the filtering is then applied by software. For example, the recorded components of the unfiltered signal are used to calculate the signal vector that would have been obtained if, for each projection, each frequency component of the signal were multiplied by a time window W(t) of a width equal to the acquisition time for that frequency, which in general may be shorter than the total acquisition time for that projection. Alternatively, the filtering of the signal is applied by hardware. For example, different frequency components are acquired by different receivers, each receiver being sensitive to a different frequency band, and for each frequency band, the signal is only recorded for the limited acquisition time that pertains to that frequency band. Alternatively, the filtering of the signal is accomplished by a combination of software and hardware.

As noted above, the acquisition time for a given frequency band component of the signal is optionally chosen depending on a characteristic magnitude of the field gradient G, and voxel width Δx, for the locations in the FOV at which the nuclear spins have a precession frequency within that frequency band. Optionally, the acquisition time as a function of frequency band is the same for all projections of the spatially encoding magnetic field. That condition may be particularly useful if the different projections represent different orientations of a magnetic field source that rigidly rotates around the FOV, because then the characteristic field gradient could be the same function of field magnitude for all projections, and if the voxel width Δx is more or less constant throughout the FOV.

The reconstructed image is optionally expressed as a vector whose components describe a weighted or unweighted magnetization of the object being imaged, for example a part of the body of a subject in the case of a medical image, at each voxel of the FOV. If the magnetization is weighted, then it is, for example, weighted by T₁, or by T₂, or by scalar or tensor diffusion weighting, or by any other type of weighting known in the art of MRI, or by any combination of those weightings. Optionally, each component of the image vector corresponds to the weighted or unweighted magnetization at a different voxel. Alternatively, each component of the image vector describes a different component of the image in k-space, or a different component of the image in some other set of basis functions, for example cubic splines, or wavelets. In any of these cases, the components of the image vector are said to describe the weighted or unweighted magnetization at each voxel of the FOV, because they can be used to calculate the weighted or unweighted magnetization at each voxel.

The reconstructed image vector describes a distribution of weighted or unweighted magnetization over the FOV that would be expected to produce the filtered received signal vector, to good approximation. For example, the distribution of magnetization would be expected to produce the filtered received signal vector to within an error no greater than a known noise level of the filtered received signal vector. The known noise level is, for example, the noise level that would be expected from known sources of electrical noise in the receiver or receivers, and optionally any additional random errors that would be introduced by signal processing, for example due to digitization of signals, or due to software that calculates the filtered signal. The noise level is characterized, for example, by the square root of the variance, due to the noise and signal processing errors, in each component of the filtered received signal vector, averaged over all the components. The error between the actual filtered received signal vector and the expected filtered signal vector that would be produced by the reconstructed image vector is defined, for example, as the square root of the mean of the squares of the differences between corresponding components of the two vectors. Optionally, the mean of the squares used here is a weighted mean, for example weighted based on an expected noise level for each component, with more weight given to components that have a lower expected noise level.

Optionally, the reconstructed image vector is an image vector that reduces, and optionally minimizes, a positive definite measure of a difference between the filtered received signal vector, and the filtered signal vector that would be expected to be produced by that image vector. For example, the expected filtered signal vector for a given image vector is an encoding matrix operating on the image vector, and finding the reconstructed image vector comprises multiplying the filtered received signal vector by an inverse or pseudo-inverse of the encoding matrix.

Alternatively, the reconstructed image vector is an image vector that minimizes the sum of a positive definite measure of a difference between the filtered received signal vector and the filtered signal vector that would be expected to be produced by that image vector, and a regularization term that depends on the image vector. Using such a regularization term, when finding the reconstructed image vector, may produce a reconstructed image vector that is more stable and less sensitive to noise in the received signal, than if no regularization term were used, because the regularization term biases the reconstructed image to have characteristics that are expected for the reconstructed image on physical grounds, for example being relatively smooth except at a few sharp boundaries between different regions. Optionally the regularization term is a Tikhonov regularization term, noise-weighted or not noise-weighted. Alternatively, the regularization term is a constant times a sum, or weighted sum, of absolute values of vector components of the image vector, or of the image vector transformed by a matrix, for example a unitary matrix. Optionally the matrix is a sparsifying transform, for example a wavelet transform. A “sparsifying transform” refers to a transform that transforms the image vector to a vector that has very low values or zero values for almost all of its components, and high values for only a few of its components, and which transforms are sparsifying depends on expected characteristics of the image. In medical MRI, and in MRI used in materials science, the image often comprises a plurality of extended regions each with uniform or nearly uniform image intensity, and sharp smooth boundaries between the regions. In such a case, a wavelet transform is potentially a good choice for a sparsifying transform.

A regularization term as described in the previous paragraph, using a sparsifying transform, may be particularly useful for implementing compressive sensing, reconstructing the image with an under sampled signal, for example with fewer projections of the spatial encoding magnetic field than would normally be needed to get good image quality without compressive sensing, for example a factor of 5 or 10 fewer projections. Such compressive sensing is possible, because the regularization term will bias the reconstructed image against having the kinds of image artifacts that would normally be found in the image if the signal is very under sampled. Without using compressive sensing, using a magnetic field source that rotates rigidly around a two-dimensional FOV by 180 degrees to produce the different projections of the spatially encoding magnetic field, the number of different projections N_{θ} needed to produce a full resolution image without image artifacts, with a uniform voxel width of Δx throughout the FOV, would be about π/2 times the ratio of the width of the FOV to Δx, times the ratio of the average magnitude of the magnetic field gradient in the FOV to an effective minimum magnetic field gradient in the FOV, with the different projections evenly distributed in rotation angle over the 180 degrees. As used herein, the effective minimum magnetic field gradient means the minimum value, for any voxel in the FOV, of an average, over all projections of the magnetic field, of the minimum average field gradient between that voxel and any of its adjacent voxels, but the effective minimum magnetic field gradient is defined as always being at least 1/10 of the average magnitude of the magnetic field gradient in the FOV, and it should be understood that if there is any part of the FOV where the actual field gradient is less than the effective minimum gradient for all or most projections, then there may be less resolution in that part of the FOV.

Optionally, finding the reconstructed image vector comprises doing a search for the image vector that minimizes the sum of the positive definite measure of the difference, and the regularization term, in the case where a regularization term is used, or doing a search for the image vector that minimizes the positive definite measure of the difference, in the case where a regularization term is not used. Optionally, the search is done using an iterative search method. Suitable exemplary search methods are described, for example, by Beck and Teboulle, “A Fast Iterative Shrinkage-Thresholding Algorithm for Linear Inverse Problems,” SIAM J. Imaging Sciences 2, 183-202 (2009).

Alternatively, the reconstructed image vector is found by calculating the image vector from an analytic expression for the image vector in terms of the filtered received signal vector. For example, if no regularization term is used, or if a Tikhonov regularization term, including a noise weighted Tikhonov regularization term, is used, then the image vector may be expressed analytically in terms of the filtered received signal vector, and the encoding matrix that transforms an image vector of the FOV into an expected filtered signal vector that would be produced by that image vector, and the Tikhonov regularization term if there is one. In these cases, the analytic expression may involve the inverse or pseudo-inverse of a large matrix.

In some embodiments, the spatially encoding magnetic field, as a function of location in the FOV, has the same form for each projection, differing only by a rigid rotation, and optionally by a rigid translation. This may be true, for example, if the magnetic field is produced by a magnetic field source or sources of a strength that does not change in different projections, for example because the field sources are permanent magnets or coils with constant current, and rotates and possibly translates rigidly, in going from one projection to the next. Alternatively, the magnetic field source comprises coils that have controllable current, that change from one projection to the next, but the currents are adjusted so that the resulting magnetic field emulates a field produced by rigidly rotating and possibly translating constant magnetic field sources. In either case, the method used for reconstructing the image from the filtered received signal takes into account that the spatially encoding magnetic field at different projections are rigid rotations and possibly translations of each other, relative to the FOV. Alternatively, the spatially encoding magnetic fields at different projections are not rigid rotations or translation of each other, but differ in shape due to different coil currents being used, and/or due to magnetic field sources that move relative to each other, at different projections.

If the spatially encoding magnetic field is produced by a rigidly rotating magnetic field source, then optionally the magnetic field source is mounted on a bearing that allows the magnetic field source to be rotated to different orientations around one or more axes, and optionally allows the magnetic field source to be moved linearly to different positions in one or more directions. Optionally, there is a motor or actuator that is configured to move the magnetic field source automatically to each orientation and position that corresponds to one of the projections. Alternatively, the orientation and/or position of the magnetic field source are adjusted manually, for some or all of their degrees of freedom.

Optionally, the magnetic field source comprises an open magnet, which is located entirely or mostly on one side of the FOV, and which rotates, optionally rigidly, around to different sides of the FOV, for different projections. Such a configuration may be useful for imaging only a portion of the body of a subject, without any need for the subject to be placed in the bore of a magnet that completely surrounds the subject, when an image is acquired, and has the potential to be much less expensive, and more portable, than a conventional MRI device. Such an open magnet is likely to produce a much more nonlinear spatially encoding magnetic field, than a conventional MRI device with a FOV inside a bore, but the use of adaptive acquisition time may nevertheless allow an open magnet MRI device to produce image of good quality, comparable to those produced by a conventional MRI device.

Optionally, the open magnet, or other magnetic field source, comprises one or more permanent magnets. For example, a magnetic cap configuration is an open magnet, using permanent magnets, that produces a spatially encoding magnetic field with relatively moderate spatial nonlinearity. A magnetic cap configuration may be particularly suitable for producing good quality images using the adaptive acquisition time method. Such a magnetic cap configuration for brain imaging is described, for example, by McDaniel et al, “The MR cap: a single sided MRI system designed for potential point-of-care limited field-of-view brain imaging,” Magn. Res. Med. 2019;00:1-15 (DOI: 10.1002/mrm.27861). A magnetic cap configuration for brain imaging might have a 3-D FOV, for example, that is between 20 and 30 cm on a side, and the magnetic cap itself might be somewhat bigger, for example 30 to 50 cm in diameter. A smaller magnet, with a smaller FOV, could be used, for example, for imaging the knee or wrist. For other kinds of medical or non-medical imaging, different sized magnets, with different sized FOVs, can be used depending on the size of the sample being imaged.

Any other open or closed magnet configuration may also be used, for example any of the open configurations described by Danielli and Blümich, “Single-sided magnetic resonance profiling in biological and materials science,” J. Mag. Res. 229, 142-154 (2013). But better image quality can potentially be obtained if the spatially encoding magnetic field isn't too nonlinear, as in the case of the magnetic cap configuration. For example, the ratio of the greatest and smallest field gradient in the FOV is greater than 5, or between 3 and 5, or between 2 and 3, or between 1.5 and 2, or between 1.2 and 1.5.

For each frequency band l, centered at frequency ωₗ, a characteristic magnitude of field gradient Gₗ may be defined. As used herein, Gₗ is defined as the mean value of the magnitude of the field gradient over the region of the FOV that has nuclear spin precession frequency within the frequency band, for a given projection of the spatially encoding magnetic field. Alternatively, other definitions of the characteristic magnitude of field gradient are used, for example a mode or median value is used instead of the mean value, and/or the mean (or median or mode) is weighted toward or limited to parts of the FOV that are expected to be of greatest interest. Optionally, if there is more than one receiver antenna, the signals from different receiver antennas are recorded separately, which can improve the quality of the reconstructed image. In this case, Gₗ may be different for the signal components coming from different receiver antennas, even for the same frequency band, and Gₗ may be chosen for a given signal component based on the field gradients at locations where the sensitivity of that receiver antenna is greater, or greater relative to other receiver antennas. Optionally, the characteristic magnitude of field gradient is defined in a way that is independent of the projection, for example the mean value is taken only over a symmetrically defined region of the FOV that is independent of the projection.

Optionally, defining Gₗ for example as the mean value of the magnitude of the field gradient over the region of the FOV that has nuclear spin precession frequency within the frequency band l for that projection, the effective acquisition time (defined for example as twice the standard deviation of Wₗ(t)) for each frequency band is positively correlated with 1/Gₗ for that frequency band, for example with a linear correlation less than 0.2. or between 0.2 and 0.5, or between 0.5 and 0.8, or greater than 0.8. Optionally, the FOV has voxels of substantially uniform width Δx, and the best linear fit of the effective acquisition time to 1/Gₗ is approximately π/(γGₗΔx), for example between 2/(γGₗΔx) and 5/(γGₗΔx), where γ is the gyromagnetic ratio for the nuclear spins.

Optionally, the spread in the widths (standard deviations) of the window functions Wₗ(t), for the different frequency bands, or for the different combinations of frequency band and receiver antenna, is a substantial fraction of the mean of the widths of the window functions Wₗ(t), for the different frequency bands, or for the different combinations of frequency band and receiver antenna. For example, the standard deviation of the widths of the window functions is at least 20% of the mean of the widths of the window functions. That would correspond to a spread in the widths of the window functions of about a factor of 1.5, since the mean value plus one standard deviation would be 1.5 times the mean value minus one standard deviation. Optionally, this is true at least for a subset of the frequency bands, or combinations of frequency band and receiver antenna, that contribute a substantial fraction of the energy of the MRI signal. For example, it is true for a subset of the frequency bands, or combinations of frequency band and receiver antenna, that contribute at least 40% of the energy of the MRI signal, or at least 20%, or at least 10%, or at least 5%. Optionally, this is true for a FOV that has a gradient that varies in magnitude by at least a factor of 1.5. In this case, the range of acquisition times (widths of time windows) for the different components of the signal may be well adapted to the range of gradient magnitude across the FOV.

Optionally, the MRI signal is received substantially only from a FOV that is one voxel thick, and the voxels are arranged in two dimensions in the FOV. For example, locations in the FOV are specified by three independent spatial coordinates, x. y. and z, not necessarily Cartesian coordinates, and the boundaries of the voxels are defined only by x and y, independent of z. Optionally, the spatially encoding magnetic field, for each of its projections, is substantially constant across the thickness of the FOV, for example the z direction, varying much less than, or at least not much more than, the spatially encoding magnetic field varies across the width of each voxel within the FOV, for example in the x or y direction, and varies less than, or not much more than, the spatially encoding magnetic field varies from one voxel to an adjacent voxel in the direction of the field gradient.

Since nuclear spins at a given location in the FOV produce an MRI signal at the precession frequency corresponding to the magnitude of the spatially encoding magnetic field at that location, these conditions on the spatial variation in the magnetic field can be made precise by expressing them in terms of the frequency distributions of the component of the MRI signal received from different voxels. For example, the nuclear spins are distributed in the FOV in such a way that, for each projection of the spatially encoding magnetic field, the standard deviation in frequency of the MRI signal received from each voxel, which depends on the distribution of magnetic field magnitude within the voxel, is less than the greatest difference in mean frequency of the MRI signal between that voxel and any adjacent voxel, which depends on the difference in mean magnetic field between that voxel and an adjacent voxel in the direction of the field gradient. Optionally, even if this is not true of all voxels in the FOV, it is true of the voxels that contribute most of the energy of the received MRI signal, for example it is true of a subset of the voxels that collectively produce at least 70% or at least 80% or at least 90% of the energy of the MRI signal, summed over the projections of the spatially encoding magnetic field. Under these circumstances, the spatially encoding magnetic field, in its different projections, can effectively distinguish between components of the MRI signal coming from different voxels, and a good image can be reconstructed from the MRI signal, with resolution comparable to the width of a voxel. Optionally, the spatially encoding magnetic field can more effectively distinguish between components of the signal that come from different neighboring voxels, than they can distinguish between components of the signal that come from the same voxel.

For example, this might happen if the object being imaged is very flat and thin, extended over a much smaller distance in the direction of the thickness of the FOV, than over the width of the FOV, with very few nuclei located outside the FOV that contribute to the signal. Alternatively, even if the object being imaged is not very thin, the nuclear spins might only be excited in a thin slice of the object. For example, there might be a gradient coil that produces a field gradient in the direction of the slice thickness only during excitation of the nuclear spins by an RF excitation pulse, but not during read-out of the MRI signal, and the excitation pulse might be narrow enough in frequency so as to excite only a relatively thin slice. Alternatively, even if the object being imaged, or the excited portion of it, is thick in the direction across the FOV, the spatially encoding magnetic field might be very uniform in that direction, over a distance covering substantially all of the object being imaged in that direction, or all of the excited part of it. For example, the spatially encoding magnetic field might be produced by a Halbach magnet, which has a magnetic field that is very uniform in the z direction and has strong gradients in the x and/or y directions. An exemplary Halbach magnet, used for MRI, is described for example by Cooley et al, “Design of Sparse Halbach Magnet Arrays for Portable MRI Using a Genetic Algorithm,” IEEE Trans. Magnetics 54, 5100112 (January 2018). In this case, the reconstructed MRI image will show the weighted or unweighted magnetization of the object being imaged, as a function of x and y, averaged over the z direction.

If the FOV is two-dimensional in this sense, and the spatially encoding magnetic field is produced by a rigidly rotating magnetic field source, then it may be possible to reconstruct the image by using a series of projections of the magnetic field produced by rotating the magnetic field source around only one axis, for example oriented perpendicular to the plane of the FOV, with the rotation angles extending over 180 degrees.

On the other hand, if, for many voxels and projections of the spatially encoding magnetic field, the standard deviation of frequency for the component of MRI signal coming from a given voxel is greater than the difference in mean frequency between the component of MRI signal coming from that voxel and all its adjacent voxels, which will generally be due to a large variation in magnetic field along the z direction within the voxel, then the spatially encoding magnetic field may not be able to distinguish as well between components of the MRI signal coming from different voxels, because they will have a large overlap in frequency distribution, and it may not be possible to reconstruct such a high resolution image. In those circumstances, a better image might be reconstructed by using voxels arranged in three dimensions.

Alternatively, the FOV is three-dimensional, with voxels arranged in three dimensions, and it is desired to reconstruct a three-dimensional image. There are several methods to accomplish this, including, for example, the following options:
1) With a rigidly rotating and/or translating magnetic field source, the different projections are optionally produced by rotating and/or translating the magnetic field source with at least two degrees of freedom. For example, the magnetic field source is rotated around two different axes oriented in different directions, or the magnetic field source is rotated around one axis, and translated along that axis.
2) With a rigidly rotating magnetic field source, the different projections are optionally produced by rotating the magnetic field source around the z axis, so that the spatially encoding magnetic field produced by the magnetic field source makes it possible to determine the x and y distribution of the signal by looking at the signal coming from the different projections. The MRI excitation pulse that excites the nuclear spins initially is optionally launched with a phase that depends nonlinearly on z. This nonlinear phase dependence of the excitation pulses makes it possible to determine the z distribution of the signal coming from each x and y. Details of how this is done, in exemplary embodiments, are described, for example, by Stockmann et al, “Transmit Array Spatial Encoding (TRASE) using broadband WURST pulses for RF spatial encoding in inhomogeneous B0 fields,” J. Mag. Res. 268, 36-48 (2016), and Jonathan C. Sharp and Scott B. King, “MRI Using Radiofrequency Magnetic Field Phase Gradients,” Mag. Res. in Medicine 63, 151-161 (2010).

Using an adaptive acquisition time is likely to provide especially great improvement in image quality, providing more uniform resolution and/or reduced aliasing image artifacts, if there is a large range of magnitude of field gradients found in the FOV, and a large range of magnitude of field gradients that contribute substantially to the image. For example, the magnitude of the field gradient varies by at least a factor of 1.2, or at least a factor of 1.5, or at least a factor of 2, over the FOV, and the effective acquisition times for two different frequency bands of the received signal, that each contribute more than 1% to a total energy of the filtered received signal, differ by at least a factor of 1.2, or at least a factor of 1.5, or at least a factor of 2. Optionally, this is true for an average of effective acquisition times for two different sets of frequency bands, where each set collectively contributes more than 1%, or more than 5%, or more than 10% to the total energy of the filtered received signal, even if it is not true of two individual frequency bands.

An aspect of some embodiments of the invention concerns an MRI device for reconstructing an image of an FOV. The device comprises the following elements:

1) A magnetic field source that produces a spatially encoding magnetic field in the FOV, that has a different dependence on position in the FOV at each of a plurality of different projections of the magnetic field. For example, the different projections could be produced by rotating the magnetic field source to different angles of orientation around the FOV, and/or by changing currents in one or more coils that the magnetic field source comprises.
2) One or more MRI excitation antennas that produce an excitation electromagnetic field, for example a pulsed electromagnetic field, that resonantly excites nuclear spins in the FOV, for each projection of the magnetic field.
3) A plurality of MRI receiver antennas, and one or more receivers that record data of an MRI signal received by each of the receiver antennas, from the excited nuclear spins in the FOV, for a total acquisition time, following the application of the excitation electromagnetic field to the FOV for each projection.
4) A signal filtering module that filters the received signal by applying time windows of different widths, which means different effective acquisition times, to at least some different frequency bands of the signal, and/or to at least some components of the signal received by different receiver antennas, resulting in a filtered received signal vector whose components describe the filtered received signal as a function of time or frequency, at each projection. As described above, the filtering can be implemented by hardware, by software, or by a combination of hardware and software, and as used herein, “signal filtering module” refers to the hardware, software, or combination of hardware and software that is performing the filtering.
5) An image reconstruction module programmed to find a reconstructed image vector whose components describe a weighted or unweighted net magnetization at each voxel in the FOV, that would be expected to produce the filtered received signal vector.

An aspect of some embodiments of the invention concerns a method and system for acquiring an MRI signal from a FOV, at each of a plurality of projections of a spatially encoding magnetic field, and using the MRI signal to reconstruct an image of the FOV, in which at least two of the projections of the spatially encoding magnetic field are distortions of each other, as opposed to being rigid translations and/or rotations of each other. For example, the spatially encoding magnetic field is produced by a magnetic field source, comprising two or more magnetic elements, including at least one magnet, and optionally the elements also include a piece of a soft magnetic material such as iron. At least one of the magnetic elements can move, rotate, or both, relative to the other element, and this can produce a distortion in the magnetic field. It is straightforward to determine what magnetic field B is produced as a function of position x by a given configuration of magnets, pieces of soft magnetic material, and other magnetic elements, and to determine if the magnetic field of one projection is a distortion of the magnetic field of another projection.

It should be understood that “move” and “motion” when referring to magnetic elements of the magnetic field source, whether rigid or non-rigid motion, refer to translation or rotation or any combination of translation and rotation, including translation which changes velocity over time, or rotation which changes its rate and/or axis over time. Optionally, one or more magnetic elements comprises a flexible material, such as the material commonly used in refrigerator magnets, or a magneto-rheological fluid, and in such a case “move” and “motion” also include deforming, bending, twisting, stretching, compressing, flowing, and any other spatial degrees of freedom of such a flexible material. The terms “position” and “orientation” when applied to such a flexible or fluid magnetic element also include state of deformation, state of bending, state of twisting, etc.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### System for Producing MRI Images Using Adaptive Acquisition Time

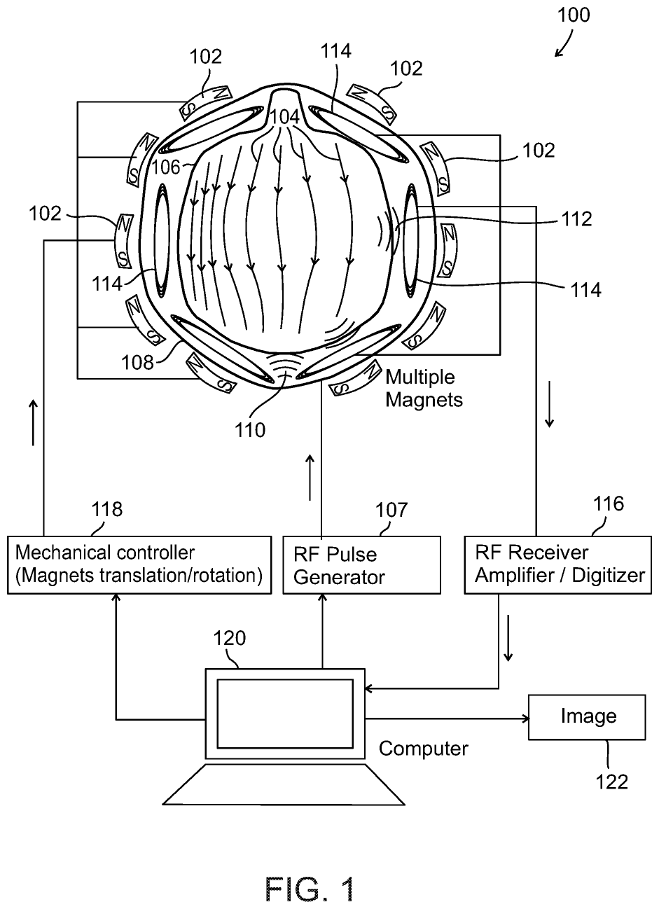

Referring now to the drawings, FIG. 1 schematically illustrates a system 100 for producing MRI images, without the need for gradient coils and without the need for a very uniform magnetic field, but capable of producing high quality images, according to an exemplary embodiment of the invention. A magnetic field source 102 comprises one of more magnets, in this case 10 permanent magnets, that produce a non-uniform spatially encoding magnetic field 104 inside a field of view (FOV) 106, which in the system shown is a person's head. The magnetic field source may also comprise soft magnetic material such as iron, and may use electromagnets instead of or in addition to permanent magnets. Electromagnets may allow higher magnetic fields than permanent magnets, but only at the cost of high power if the electromagnets are made of conventional conductors, or making the system expensive if superconducting electromagnets are used.

If the magnetic field source does use electromagnets, then optionally the current in the electromagnets does not change during each projection of the spatially encoding magnetic field, which has the potential advantage that it is simpler to determine the encoding matrix that transforms a given image vector into an expected signal vector that it would produce, and that is used in reconstructing the image from the signal. Optionally, the current in the electromagnets does not change during the entire time that the system is producing an image of the FOV, which has the potential advantage that it may be easier to keep the spatially encoding magnetic field at a known value as a function of position in the FOV, and the magnetic field source may be less complicated.

Optionally, the spatially encoding magnetic field has a maximum value, in the FOV, that is lower than typical fields used in the bores of conventional MRI devices, which typically have fields between 1 and 3 tesla. For example the maximum field in the FOV is less than 0.2 tesla, or between 0.2 and 0.3 tesla, or between 0.3 and 0.5 tesla, or between 0.5 and 0.7 tesla, or between 0.7 and 1.0 tesla. Lower magnetic fields have several potential advantages, including the possible use of permanent magnets or conventional coils, as opposed to expensive superconducting coils; lower weight and more portability of the system; and the possibility that there might be less distortion of the magnetic field, and consequently less distortion of the reconstructed image, due to effects of the magnetic susceptibility of the object being scanned. For a system with a moderately large FOV, for example a system designed to scan a human brain or a human knee, using a maximum field in the FOV that is greater than 0.3 tesla may make the system heavy enough so that it will not be easily portable.

In the example shown in FIG. 1, spatially encoding magnetic field 104 has a higher intensity on the left side of the head than on the right side, which could be accomplished, for example, by having stronger magnets on the left side of the head than on the right side. The magnetic field has a vector value B(x), where the vector x represents the position in the FOV. An RF pulse generator 107 synthesizes a sequence of pulsed RF signals that are fed to one or more transmitting antennas 108, in this case a single circular antenna that surrounds the head. The sequence could be any standard MRI pulse sequence. Antenna 108 produces an RF field 110 throughout FOV 106, polarized so that it has a component of RF magnetic field perpendicular to B, and it causes the spins of the nuclei of hydrogen atoms in the FOV, initially oriented in the direction of B, to acquire a component of spin perpendicular to the direction of B, and to precess around the B direction at the local magnetic resonance frequency, which is γB(x), where γ is the gyromagnetic ratio of the nuclei, about 42 MHz/T for hydrogen. The precession of the nuclei produces an RF magnetic field 112, with frequency components at the precession frequency of each precessing nucleus, and also oriented perpendicular to the B direction. This combined MRI signal from the precessing nuclei is detected by one or more RF receiver antennas 114, in this case six receiver antennas arranged around the head. The receiver antennas send their signals to an RF receiver 116, which amplifies the signal from each receiver antenna, and digitizes it and records it. The signals from at least some of the different antennas are kept separate and converted into its own digitized signal and recorded. Optionally, the signals from all the different antennas are recorded separately. Alternatively, the signals from one or more receiver antennas are combined at the receiver, and only the combined signal is digitized and recorded. It is should be understood that, in a case where the signals from a set of two or more receiver antennas are combined and only the combination is recorded, terms such as “signal components received by each receiver antenna” can refer also to signal components that are a combination of signals received by such a set of receiver antennas. Optionally, receiver 116 also Fourier analyzes the signal into a plurality of relatively narrow frequency bands, and digitizes and records each frequency component as a function of time. Each frequency component of the signal represents the signal received from nuclei in locations with a narrow range of values of B(x).

The signal received by a given antenna, or combined set of antennas, from a given location x in the FOV is proportional to the density of hydrogen atoms at that location, and to the spatial sensitivity c(x) for that antenna or combined set of antennas, possibly weighted by T₁, T₂, a diffusion coefficient, and/or any other MRI parameter. Specifically, the signal is proportional to the net magnetization density m(x) of nuclei at each location, times c(x). The relation of m(x) to the density of hydrogen atoms at x, and to the various MRI weighting parameters, depends on the timing, time duration, amplitude and phase of the pulses in the pulse sequence. Although any known MRI pulse sequence could be used, the inventors believe that a spin echo sequence, with short T_{E} (time to echo), for example less than 1 millisecond, for example a Carr-Purcell-Meiboom-Gill (CPMG) pulse sequence, optionally with frequency sweeping, may be especially suitable for a system such as system 100, which has high field gradients and a wide range of field strengths B over the FOV. Using a short echo time has the potential advantage that it may better allow a coherent echo to form even when there is a wide range of frequencies, corresponding to a wide range of values of B, contributing to the echo. Using frequency sweeping has the potential advantage that there may be a smaller range of frequencies contributing to each echo, which may also better allow a coherent echo to form.

Once the MRI signal has been received, a mechanical controller 118 causes the magnets or other elements of magnetic field source 102 to move, for example to translate and/or rotate, to a new position. If the magnetic field source also includes electromagnets, the current in one or more of the electromagnets may change to a new value, in addition to or instead of the magnets and other elements of the magnetic field source moving. However, it is potentially advantageous not to have any electromagnets in the magnetic field source, or not to have electromagnets that have varying current, since they would play the role of gradient coils in a conventional MRI device, and might make the system more expensive, less portable, and/or more power consuming. At the new positions and orientations of the magnets and any other elements, the spatially encoding magnetic field B(x) in the FOV will be different than before. This is a different projection of the magnetic field in the FOV. Optionally, the magnetic field has the same shape as before, and is only rigidly translated and/or rotated in the new projection. This could be accomplished, for example, by only using permanent magnets, or electromagnets with constant current, and optionally iron, and rigidly rotating or translating the whole magnetic field source together. Alternatively, the magnetic field is distorted in going from one projection to another, for example by having one magnet or other element of the magnetic field source move or rotate relative to another magnet or other element, in going from one projection to another.

At the new projection, an MRI pulse sequence is produced again in the FOV, and the MRI signals are received and digitized and recorded again. This may be repeated many times, for example rotating the magnetic field source, or some of the elements of the magnetic field source, by 180 degrees in 1 degree increments for each new projection, receiving the MRI signal for each projection. The combined MRI signals from all the projections provide information that may be used to reconstruct an image.

A computer 120 receives the digitized MRI signals, for each projection and for each receiver antenna (if saved separately) from receiver 116, and records the signals in its memory. Computer 120 also communicates with mechanical controller 118, telling it when and how to move the elements of the magnetic field source to produce a new projection of the spatially encoding magnetic field, and communicates with RF pulse generator 107, telling it the timing, time duration, amplitude and phase of each of the pulses of the pulse sequence, and when the pulse sequence should be initiated. Computer 120 records the timing of the received MRI signals based on the timing of the pulse sequences. Finally, after the MRI signals have been received and recorded for all the projections, computer 120 reconstructs an image 122 of the FOV, using the MRI signals, as will be explained. It should be understood that these different functions of computer 120 can be performed by different software modules running on a single computer, for example a personal computer with appropriate I/O interfaces. Alternatively, two or more physically separate computers or controllers, in communication with each other, can perform these different functions. In that case the combination of separate computers or controllers is referred to as computer 120.

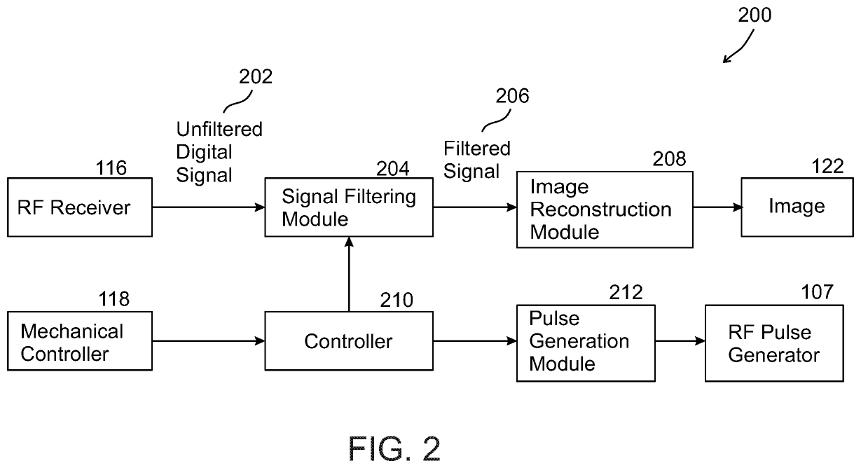

FIG. 2 shows a block diagram 200, illustrating the different computer modules that perform the functions of computer 120 in FIG. 1, and their communication links to other elements shown in FIG. 1, according to an exemplary embodiment of the invention. It should be understood that these “computer modules” may comprise programmed software modules that are running on a general purpose computer, or may comprise circuitry dedicated to performing the specified functions, or any combination of hardware, firmware, and software. RF receiver 116 optionally sends unfiltered digitized MRI signals 202 to a signal filtering module 204 running on computer 120. Signal filtering module applies different signal acquisition time windows to components of the signal that come primarily from locations with different magnetic field gradients, for example to different frequency components of the MRI signal, and/or to components from different receiver antennas. The signal filtering module produces as output a filtered signal 206, expressed for example in the time domain or in the frequency domain, for each projection, and for each receiver antenna for which the signals are saved separately. Alternatively, instead of applying different acquisition time windows to the different signal components by software running on computer 120, the acquisition time windows are applied to the signal by hardware, for example by hardware incorporated into RF receiver 116, or by a combination of hardware and software. For example, RF receiver 116 may only record the signal components for the duration of the time window for each frequency component and/or receiver component. This kind of filtering may be particularly easy to implement in hardware if different receiver antennas are sensitive to different frequency bands of emitted MRI signal 112. In either case, the filtering results in a filtered signal 206 as output.

Optionally, the signal filtering module filters the signal for only some of the projections, not all of them, and the signal filtering module outputs the unchanged signal for the projections for which the signal is not filtered, but the total output of the signal filtering module, including the output for all the projections, is still referred to as the filtered signal.

Filtered signal 206, for all the projections and receiver antennas, is sent to an image reconstruction module 208 running on computer 120, and the image reconstruction module generates a digital image file 122 as output. The image file is optionally displayed on a screen, printed it out, and/or stored in a digital recording medium, for example as part of the patient's medical record.

A controller or executive module 210 optionally controls and coordinates the different software modules of computer 120, for example controlling the timing of producing the pulse sequences, receiving the MRI signal, and moving the elements of the magnetic field source to a produce a new projection. Controller 210 optionally tells signal filtering module 204 what acquisition time windows to use for the different frequency components and/or receiver antenna components of the MRI signal for each projection, if the filtering is done at least partly with software. Alternatively, if the filtering is done entirely with hardware by RF receiver 116, this information may be, for example, stored as firmware in RF receiver 116, or may be communicated to RF receiver 116 directly by controller 210. The shape, timing, time duration, amplitude and phase of the pulse signals are optionally generated by a pulse generation module 212, under the control of controller 210, which communicates these parameters of the pulse sequence to RF pulse generator 107. Controller 210 also optionally controls mechanical controller 118, so that, for example, a new pulse sequence is initiated, and RF receiver prepares to receive and digitize a new MRI signal, after the elements of the magnetic field source have been positioned for each new projection.

### Alternative Magnet Field Source Configurations

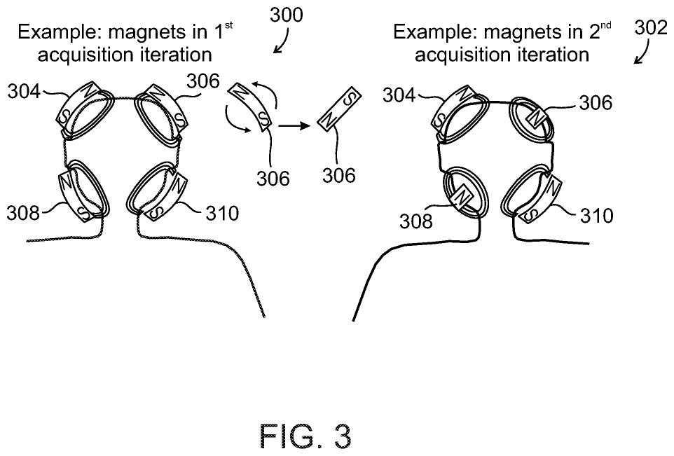

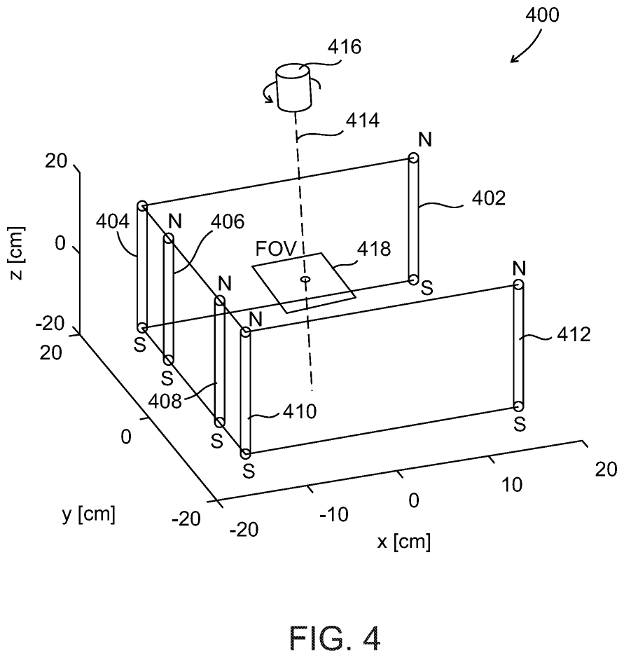

FIG. 3 shows an alternative exemplary configuration for the magnetic field source and the RF receiver antennas, to that shown in FIG. 1, Two configurations 300 and 302 are shown, corresponding to two different projections of the spatially encoding magnetic field. Configuration 300 shows four permanent magnets, magnet 304, magnet 306, magnet 308 and magnet 310, arranged around a subject's head. The direction of magnetization (the long direction) of each magnet is oriented parallel to the adjacent surface of the head, in the plane of the drawing, with the north pole of each magnet oriented toward the top of the head. Between each magnet and the head is a receiver antenna, for example circular in shape, in a plane approximately parallel to the surface of the head under the magnet, and each of these receiver antennas is optionally much closer to one of the magnets than to the other magnets. In configuration 302, two of the magnets, magnet 306 and magnet 308, have been rotated by 90 degrees, so they are oriented perpendicular to the plane of the drawing, still parallel to the adjacent surface of the head, with the north poles pointing toward the viewer of the drawing. The rotation is done, for example, by a motor or actuator that rotates both magnets, or by a separate motor or actuator that rotates each magnet. The motor or actuator is not shown in FIG. 3, but may be similar to motor 416 that rotates the configuration of magnets in FIG. 4. The other two magnets, magnets 304 and 310, remain in the same orientation as in configuration 300.

Because the magnets in configuration 302 are not all oriented in the same direction, relative to each other, as the magnets in configuration 300, the spatially encoding magnetic field produced by the magnets in configuration 302 will not be a rigid rotation and translation of the spatially encoding magnetic field in configuration 300, but the spatially encoding magnetic field in configuration 302 will be distorted relative to the spatially encoding magnetic field in configuration 300.

Optionally, other projections of the spatially encoding magnetic field, for the system shown in FIG. 3, are produced by rotating magnets 306 and 308 by a same angle different from 90 degrees, for example by a series of smaller increments, for example between 0 and 180 degrees, while the other two magnets remain in the same orientation. This would produce a series of projections with changes in one degree of freedom, which might be suitable, for example, for reconstructing an image of a 2-D FOV. Alternatively, if magnets 306 and 308 are both rotated by a first angle, while magnets 304 and 310 are both rotated by a second angle, independent of the first angle, then a series of projections would be produced with two degrees of freedom, which might be suitable for reconstructing an image of a 3-D FOV. Any other selections of the four magnets could also be chosen to rotate by a first angle, by a second angle, or not rotating at all, to generate a series of projections of the spatially encoding magnetic field with one or two degrees of freedom, which might be suitable for reconstructing an image of a 2-D or 3-D FOV.

Optionally, in addition to or instead of moving or rotating magnets in the system of FIG. 3 to produce different projections of the spatially encoding magnetic field, magnetic elements made of iron or another soft magnetic material are moved or rotated, which can also distort the magnetic field.

The configuration shown in FIG. 3, with each receiver antenna located close to only one of the magnets, may be especially advantageous when components of the MRI signal from the different receiver antennas are recorded separately and used to reconstruct the image. The magnitude B of the magnetic field in the FOV is likely to be greater near each magnet, and to be lower further away from each magnet, but nuclei at the same B, close to different magnets, will produce a signal component at the same frequency. If each receiver antenna is sensitive mostly to signals coming from the vicinity of only one magnet, and much less sensitive to signals coming from the vicinity of other magnets, this may help to reconstruct a better quality image of the whole FOV. Optionally, the magnetic field source comprises a plurality of magnets, there are a plurality of receiver antennas, and at least some of the receiver antennas are each located much closer to a different one of the magnets than to the other magnets, and the signal received by the different receiver antennas are recorded separately.

If the different magnets have different sizes, shapes, and magnetizations, then the relation magnetic field B and field gradient G may be different in the vicinity of the different magnets, and it is potentially useful, in implementing adaptive acquisition time, to use a different acquisition time window, as a function of frequency, for signal components coming from different receiver antennas that are close to different magnets.

FIG. 4 shows a U-shaped magnet configuration that can be used as the magnetic field source in a system like system 100 of FIG. 1, to generate a series of projections of a spatially encoding magnetic field, for reconstructing an image of a 2-D FOV. This magnet configuration was used to perform some of the simulation tests of the reconstruction algorithm described in the Examples section. The U-shaped magnet configuration was simulated by six long, thin permanent magnets, labeled 402, 404, 406, 408, 410, and 412. Each magnet has cross-sectional area A much less than the square of its length L, and has magnetic permeability μ, relative to the vacuum permeability μ₀, much greater than L²/A. In these circumstances almost all of the flux of the magnet will emerge from near the top and bottom surfaces of the magnet, and the spatially encoding magnetic field can be calculated to good approximation by replacing each magnet by a north magnetic monopole at its north pole, which is the top of each magnet in FIG. 4, and by a south magnetic monopole of the same magnitude at its south pole, which is on the bottom of each magnet in FIG. 4. In reality, the flux from the north pole of each magnet goes through the length of the magnet and connects to the south pole. In the simulation described in the Examples section, the “magnetic charge” of the “monopoles,” which is just the total flux going through the magnet, is the same for magnets 402 and 412, and is half that great for magnets 404, 406, 408, and 410. Each of the magnets is 20 cm long, extending from z=−10 cm to z=+10 cm. In the projection shown in FIG. 4, which could be the initial projection for example, magnet 402 is located at x=+15 cm and y=+15 cm. Magnet 404 is located at x=−15 cm and y=+15 cm. Magnet 406 is located at x=−15 cm and y=+7.5 cm. Magnet 408 is located at x=−15 cm and y=−7.5 cm. Magnet 410 is located at x=−15 cm and y=−15 cm. Magnet 412 is located at x=+15 cm and y=−15 cm.

The magnets can be viewed as joined rigidly together, and attached to a framework that rotates rigidly around the z-axis 414. This system could be implemented by using a motor 416 to rotate the system of magnets by different angles from the initial orientation shown in FIG. 4, to produce the different projections of the spatially encoding magnetic field. Because all of the magnets rotate rigidly together, the spatially encoding magnetic field does not distort in going from one projection to another, as happens in the system shown in FIG. 3, but keeps the same shape, rotating rigidly with the magnets. Because there is only one degree of freedom, the rotation angle around the z-axis, in the different projections, this system may be most useful for a two-dimension FOV, though it could also be used for a three-dimensional FOV if another method, not depending on the spatially encoding magnetic field, is used to distinguish signals coming from different 2-D slices of the 3-D FOV.

A two-dimensional field of view 418 is square, located at z=+1 cm, and extending between x=+5 cm and −5 cm, and between y=+5 cm and −5 cm. This is centered near the x-axis, where the magnetic field gradient, predominantly in the x-direction, has a local maximum, at the projection for which the magnets are located as shown in FIG. 4. The field gradient is only weakly dependent on y in the FOV, and is lower for x>0 or x<0. The magnetic field is predominantly in the z-direction in the FOV. At other projections, the magnets are rigidly rotated together around axis 414 from the positions shown in FIG. 4, and the magnetic field as a function of position, relative to the x-y-z coordinate system and the FOV, is simply rotated around axis 414 by the same angle as the magnets.

In the simulations done with this magnet configuration, described in the Examples section, the spatial sensitivity of the receiver coil was set equal to 1 everywhere. This could be implemented, for example, by using a closed loop transmission antenna and a closed loop receiver antenna in any plane that includes the z-axis, with the magnets being near the center of a transmission antenna and a receiver antenna that are at least a few times bigger in diameter than the dimensions of the magnets and the distance between them. Such a transmission antenna will generate an RF magnetic field that is nearly perpendicular to the z-direction, and the preces sing nuclei will emit RF waves that have magnetic fields that are nearly perpendicular to the z-direction, that the receiving antenna will be sensitive to. The magnitude of the transmitted RF magnetic field, and the received field, will be only weakly dependent on location within the FOV. In practice, greater detection sensitivity, for a given transmission power, might be achieved if a smaller transmission and receiver antenna are used, closer in to the FOV, but then the sensitivity c(x) will not be uniform, and will have to be taken into account in reconstructing the image.

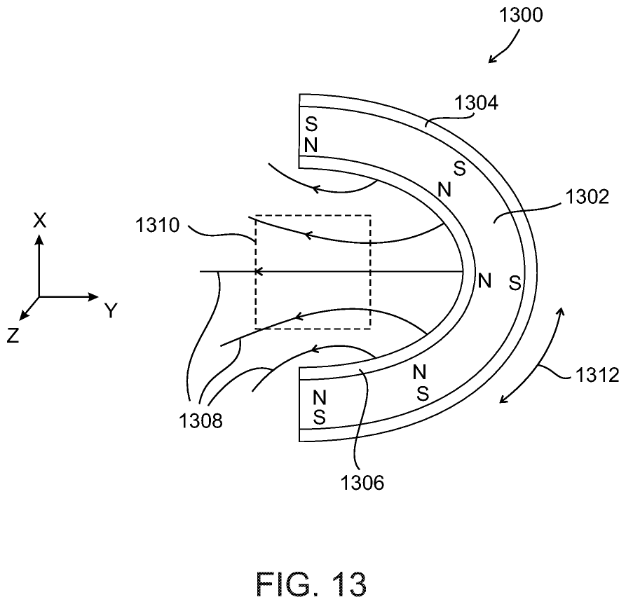

Another magnet configuration used for simulations described in the Examples section, using a cap-shaped magnet, is shown in FIG. 13, which will be described in the Examples section.

### Method of Acquiring MRI Signals Used to Reconstruct Image

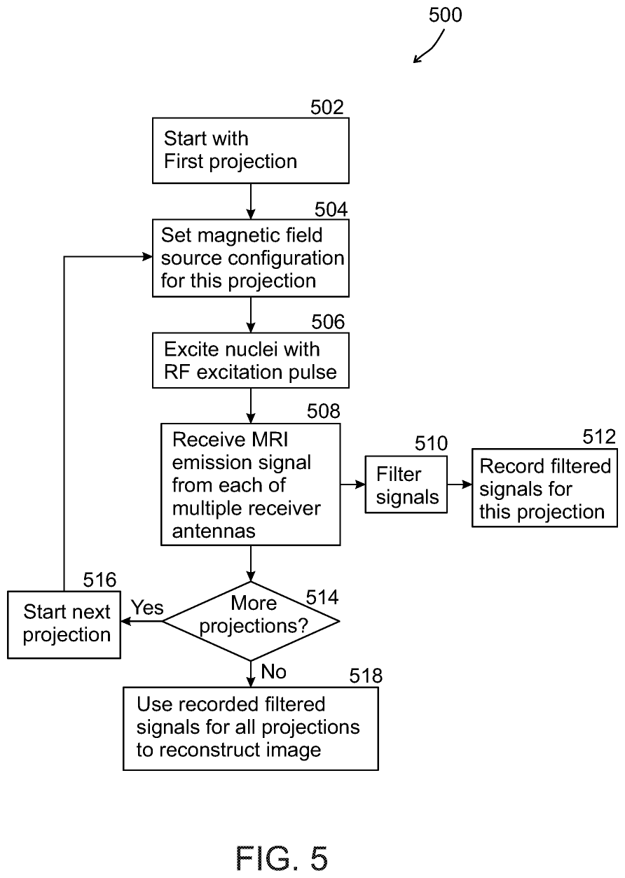

FIG. 5 shows a flowchart 500 for a method of scanning an object in a FOV, by acquiring MRI signals, used to reconstruct an MRI image, using adaptive acquisition time, according to an exemplary embodiment of the invention. The method starts at 502, with the first projection of the spatially encoding magnetic field. At 504, the magnetic field source configuration, which comprises the location and orientation of the one or more magnets, and optionally other elements made of soft magnetic material such as iron, is set to produce the spatially encoding magnetic field corresponding to the current projection, which initially is the first projection. If the magnets include any electromagnets with variable current, then the magnetic field source configuration may also include the values of the current for those electromagnets.

Optionally, the spatially encoding magnetic field B(x) has a magnitude B(x) that is monotonic in the FOV, and there are no locations x in the FOV for which the gradient of B vanishes, for any of the projections. Generally, this may make it possible to unambiguously reconstruct the image, the magnetization m(x) for the whole FOV, using the received MRI signals for all the projections, even if there is only one receiver antenna, or if there is more than one receiver antenna but the MRI signals from all of the receiver antennas are combined and not recorded separately. However, if there are multiple receiver antennas, and the received signals from at least some different antennas are recorded separately, then it may be possible to unambiguously reconstruct the image even if the projections have B(x) that is not monotonic, and even if there are locations where the gradient of B vanishes. This is a potential advantage of using multiple receiver antennas and recording their signals separately.

At 506, nuclei in the FOV are excited by a sequence of RF excitation pulses generated the transmission antenna, for example antenna 108 in FIG. 1. Any known pulse sequence used in MRI, for example an unweighted pulse sequence that only measures the density of hydrogen nuclei, or a weighted pulse sequence designed to measure one or more MRI parameters such as T₁, T₂, T₂*, or diffusion, may be used. The inventors believe that a spin echo pulse sequence with short time-to-echo T_{E}, for example echo time less than 1 millisecond, for example a CPMG pulse sequence, optionally with frequency sweeping, may be particularly suitable for this kind of system, with large field gradients and a large range of B values in the FOV.

At 508, the MRI signal is received from each of the one or more receiver antennas. At 510, the MRI signal is filtered, by limiting the acquisition time, for example using a time window, on different components of the MRI signal, for example different frequency components, and/or components of the MRI signal from different receiver antennas, using different acquisition times, or time windows of different widths, for different components of the signal. Specifically, components of the signal that come from parts of the FOV with higher magnetic field gradient are given a shorter acquisition time or narrower time window, and components of the signal that come from parts of the FOV with lower magnetic field gradient are given a longer acquisition time or wider time window. As noted, the signal can be filtered either by hardware, only recording a component of the signal for a limited acquisition time, or by software, truncating the digital signal in time or applying a time window to it as part of digital signal processing, or by a combination of hardware and software. At 512, the filtered signal is recorded for this projection.

At 514, it is determined if there are more projections for which the signal will be collected. If there are, then at 516 the next projection is started, and the magnetic field source configuration is set for the new projection. At 504, the magnets and any other elements of the magnetic field source are moved to their new positions and/or orientations, for example using motors or linear actuators, to produce the spatially encoding magnetic field for the new projection. To find the magnetic field B(x) in the FOV for each projection, which is used for reconstructing the image, any standard magnetic design software, for example finite element software, may be used, using the known positions and orientations of the magnets and any other elements, for each new projection.

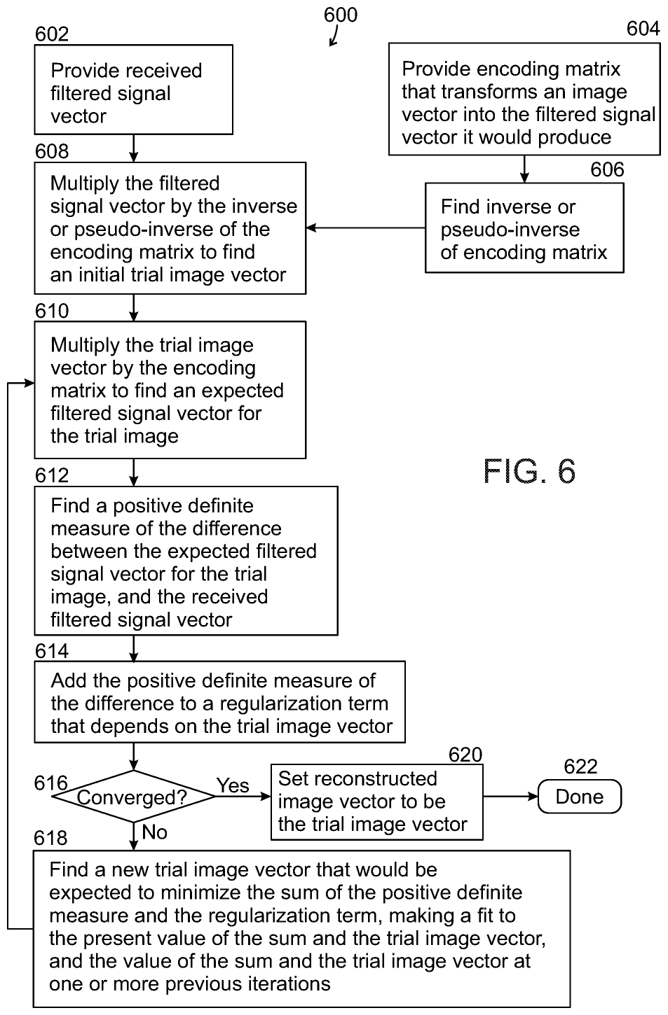

If there are no more projections that are to be used, then at 518 the recorded filtered signals for all the projections are used to reconstruct the image. Details of how this is done are shown in flow chart 600 of FIG. 6.

In some embodiments of the invention, reconstructing the image makes use of an encoding matrix, which depends on the accurately known form of the spatially encoding field B(x) at each projection. The form of B(x), produced by a known configuration of magnetic elements of the magnetic field source, is optionally determined using standard magnetic field software, such as magnetic finite element software. If the form of B(x) is not known accurately for all the projections, this may cause errors in the reconstructed image. Optionally, in order to ensure that B(x) is know accurately for each projection, the following calibration procedure is used, before scanning an object. For example, the calibration procedure may be used occasionally during idle time of the system, or periodically as part of a maintenance procedure. The calibration procedure optionally comprises:
1) placing in the FOV a phantom that produces a known image;
2) acquiring an MRI signal from the FOV for each of the projections that will be used for scanning the object to be scanned by the system;
3) reconstructing an image of the phantom from the MRI signal acquired at each projection;
4) comparing the reconstructed image of the phantom with the known image of the phantom; adjusting one or more components of the encoding matrix, based on differences between the reconstructed image of the phantom and the known image of the phantom, to values that would make the reconstructed image of the phantom more closely match the known image of the phantom; and
6) replacing the phantom in the FOV with the object to be scanned.
   Optionally, adjusting the components of the encoding matrix is done iteratively, at each iteration checking the difference between the reconstructed image of the phantom and the known image, for example until the difference is smaller than some pre-set tolerance. Optionally, the iterative procedure is performed using any known method of iteratively solving an over-determined large matrix equation.

The MRI signal s as a function of time t, received for a given projection from a given receiver antenna, may be expressed as:

s_{θ}(t)=∫B_{θ}²(x)cos(φ_{θ}(x))c(x)m(x)exp(iγB_{θ}(x)t)dx (1)

Here the subscript θ labels the projection. For example, for the system shown in FIG. 4, the different projections correspond to rigid rotations of the magnets around axis 414, and θ could represent the rotation angle of a projection, relative to the position of the magnets shown in FIG. 4. But in general, the different projections may be produced by motion of the magnets other than rigid rotation around one axis, for example the magnets may rotate around more than one axis, and/or the magnets may move relative to each other, rather than moving rigidly together, produce a distortion in the magnetic field at different projections, and the magnets may include electromagnets which have different currents at different projections. In all these cases, Eq. (1) may still be valid, and θ may be considered an arbitrary label of the projection. B_{θ}(x) is the magnitude of the spatially encoding magnetic field B at location x for projection θ. The angle φ_{θ}(x) is the angle between B and the orthogonal to the plane of the electric and magnetic fields of the RF excitation pulse. Only the component of the excitation magnetic field that is perpendicular to B contributes to the precession of the nuclei and to the MRI signal. The function c(x) is the sensitivity of the receiver antenna as a function of location x, and it should be understood that if there is more than one receiver antenna, then c(x) is different for different receiver antennas, though it is not explicitly labeled by receiver antenna in Eq. (1). The net magnetization of the nuclei, at the echo time, t=0, as a result of the excitation, is m(x), and that is the image that it is our goal to reconstruct from the MRI signals. As noted previously, γ is the gyromagnetic ratio for the nuclei, 42 MHz per tesla for hydrogen.

The factor of B_{θ}² in Eq. (1) is due to two different effects, each of which produces a factor of B in the signal amplitude coming from a nuclei at a given location x. The first effect is that the population difference between spin-up and spin-down nuclei placed in a magnetic field has a good approximation as a linear function of B. The second effect is that according to Faraday's law, the current induced in the RF receiver coil is directly proportional to the Larmor frequency, ω=γB, because the time derivative of the magnetic flux passing through the coil increases linearly with ω.

Henceforth, for simplicity, the subscripts θ labeling the projection will not be shown, but it should be understood that B and φ depend on the projection θ.

The MRI signal as a function of frequency ω may be found by taking the inverse Fourier transform of Eq. (1).

$$P ( ω ) = \frac{{ω}^{2}}{{γ}^{2}} \int cos ( φ ( x ) ) c ( x ) m ( x ) δ ( γ B ( x ) - ω ) dx \qquad ( 2 )$$

This equation shows that the contribution to the MRI signal at a frequency ω comes from nuclei located at positions x for which γB(x)=ω.

Equations (1) and (2) can be used to estimate how many different samples of the signal are needed as a function of time t, and how many different projections θ are needed, in order to resolve the image over the FOV to a feature size δₓ, which may be considered a pixel width. This estimate will be illustrated here for the case where there is only one receiver antenna, where the minimum desired feature size δₓ is independent of x, and where the projections θ represent rigid rotations of the magnetic field around one axis. The accumulated phase in the signal since t=0 is defined as ϕ(x, t)=γtB(x). A local wavenumber k(x, t) is defined as the gradient of the accumulated phase:

kₓ(x,t)=∇ₓϕ(x,t)=γt∇ₓB(x)=γtG(x) (3)

Here G(x) is the gradient of the field magnitude B(x). In order to resolve features of size δₓ in m(x) everywhere in the FOV in the reconstructed image, the signal should be acquired over a long enough time, between t=−tₘₐₓ/2 and t=+tₘₐₓ/2, so that wavenumbers up to kₘₐₓ=π/δₓ in magnitude contribute to the signal. Then tₘₐₓ=kₘₐₓ/γGₘᵢₙ=π/δₓγGₘᵢₙ, where Gₘᵢₙ is the lowest magnitude of field gradient G(x) anywhere in the FOV. This result assumes that B(x), c(x) and φ(x) are changing as a function of position x on a distance scale much greater than the smallest features δₓ that we want to resolve in m(x). To resolve a local feature in the vicinity of a position x with a size δₓ, the acquisition time should be tₘₐₓ=π/δₓγG(x), where G(x) is the local magnitude of the field gradient, assumed not to change very much over the width δₓ of the feature.

The MRI signal is optionally sampled at time intervals Δt no greater than 1 divided by the bandwidth, which is 1/γΔB, where ΔB is the range of B across the FOV. Defining Nₓ=(width of FOV)/δₓ, which may be thought of as the number of pixels across the FOV, and defining the average field gradient G_{avg}=ΔB/(width of FOV), the number of times Nₜ at which the signal should be sampled, over a time of tₘₐₓ, will be NₓG_{avg}/Gₘᵢₙ. If B(x) has a gradient G(x) that varies in magnitude by a large factor across the FOV, then the signal may be sampled many more times than in conventional MRI, where the field gradient G(x) is very uniform, to obtain the same resolution.

To estimate how many projections are to be used, to resolve features of width δₓ in the image over the whole FOV, we consider the case where the different projections are produced by rigid rotations of the spatially encoding magnetic field around a single axis, at equal angular intervals Δθ. Then Δθ should be less than 2/Nₜ. If the total rotation is over 180°, then the number Nᵣ of different rotations (projections) needed is π/Δθ=(π/2)Nₜ. It should be noted that the number of different rotations (projections) is sometimes referred to herein as N_{θ}, and the expressions Nᵣ and N_{θ} are used interchangeably. Like Nₜ, this number is greater by a factor of G_{avg}/Gₘᵢₙ than the number of projections that would be needed in conventional MRI where G(x) is very uniform. If projections with two degrees of freedom were used to reconstruct an image for a three-dimensional FOV, for example rotating the spatially encoding magnetic field around two different axes, then the number of projections along the second degree of freedom might also be greater, by a factor of G_{avg}/Gₘᵢₙ, than for conventional MRI.

This increase in the total number of signal samples taken over all projections may result in heavier computation requirements and/or longer scan times, for a given image resolution, and may be considered a cost of using an inexpensive portable MRI system with open magnet and no gradient coils. As will be described, however, the increased number of signal samples may be compensated, at least in part, by using compressed sensing, which may take advantage of the fact that medical MRI images are known to often consist of relatively uniform regions, representing different tissues, with sharp boundaries between them.

### Effect of Acquisition Time on Image Quality

As noted above, a signal acquisition time of tₘₐₓ, i.e. from t=−tₘₐₓ/2 to t=+tₘₐₓ/2, will resolve a feature of width δₓ, localized around a position x, if tₘₐₓ=π/δₓγG(x). A shorter acquisition time will result in worse spatial resolution of the feature. However, using an acquisition time longer than tₘₐₓ=π/δₓγG(x) may cause aliasing artifacts in the image. If G(x) varies very much across the FOV, then there may be no one acquisition time that gives good resolution everywhere and avoids aliasing artifacts everywhere. In that case, the best image quality may be obtained by using different acquisition times for different components of the signal, that come from locations with different magnitude of the field gradient G(x). Even if the magnitude of the field gradient does not vary very much across the FOV, it may still be advantageous to use different acquisition times for components of the signal that come from different locations where different image resolutions are needed, or where features are expected to have different characteristic sizes.

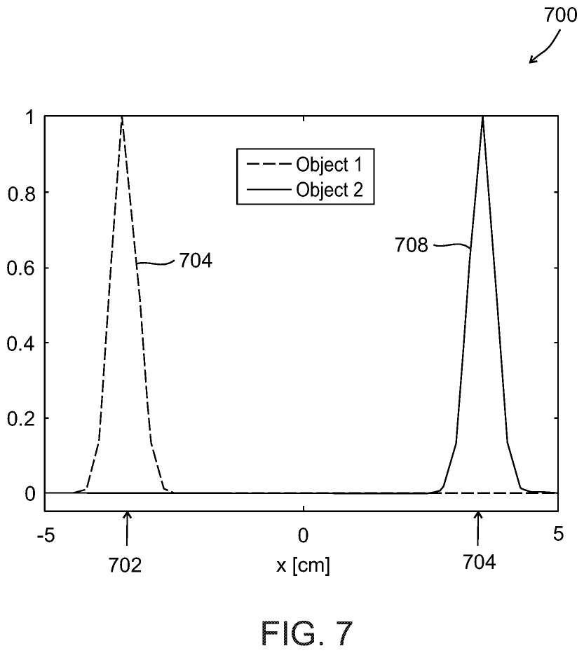

To illustrate this situation, consider FIG. 7, which schematically shows a plot 700 of magnetization m(x) for two compact objects at different locations in a 1-D FOV. Object 1 is localized around point 702 in the FOV, at x=−3.7 cm, and object 2 is localized around point 704, at x=+3.7 cm. The magnetization of object 1, as a function of x, is given by curve 706, and the magnetization of object 2 is given by curve 708. Each object is about 1 cm wide, and it is desired to image them to that resolution. The magnetic field gradient G(x) is assumed to be twice as great in the vicinity of point 702 as it is in the vicinity of point 704. It is assumed that G(x) is varying relatively smoothly with x, and does not change very much within the width of object 1 or within the width of object 2.

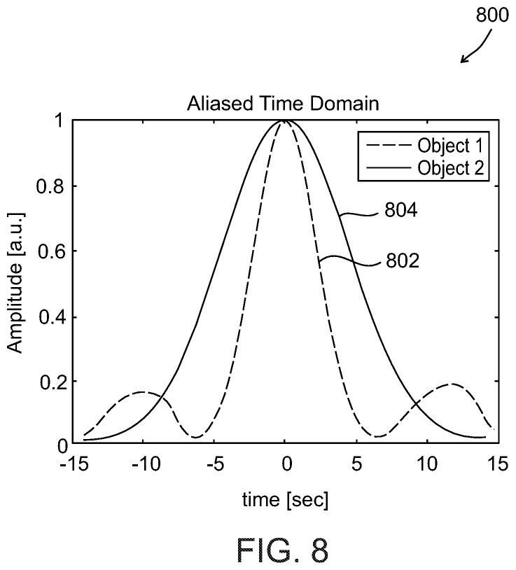

Because the field gradient is twice as great for object 1 as for object 2, and because the desired imaging resolution δₓ is the same for both objects, tₘₐₓ is half as great for object 1 as it is for object 2. In this example, tₘₐₓ is 15 seconds for object 1, and 30 seconds for object 2. FIG. 8 shows a schematic plot 800 of the MRI signals as a function of time for object 1 and object 2, signal 802 for object 1 and signal 804 for object 2. The two signals can be cleanly separated from each other, and plotted separately, because B(x) is very different for the two objects, and the ranges of B(x) for the two objects do not overlap, so the two signals are centered at different frequencies and do not interfere with each other. The plots in FIG. 8 are not necessarily based on an exact calculation using the m(x) profiles shown for object 1 and object 2 in FIG. 7, but show qualitatively what the absolute values of the signals will look like as a function of time, for the two objects. In FIG. 8, signal 804, for object 2, falls almost to zero at its ±tₘₐₓ/2, which is ±15 seconds for its field gradient. Signal 802, for object 1, falls almost to zero at its ±tₘₐₓ/2, which is ±7.5 seconds for its higher field gradient and starts to rise again at t greater than 7.5 seconds and less than −7.5 seconds. This rise of the signal beyond ±tₘₐₓ/2 may be caused by the sharp cutoff in m(x) for object 1 at its edges, and may cause aliasing artifacts in the reconstructing image, if this part of the signal is used in the image reconstruction.

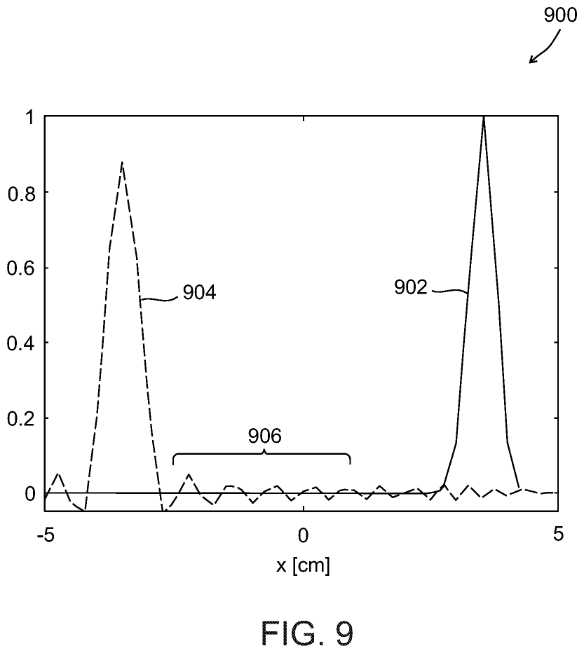

FIG. 9 shows a schematic plot of 900 of what the reconstructed image would look like, based on the MRI signals for object 1 and object 2 show in FIG. 8, with the acquisition time extending out to ±15 seconds. Reconstructed image 902 shows object 2. It looks similar to the original object, labeled 708, in FIG. 7, with its width fully resolved, and without aliasing artifacts, because its MRI signal 804 in FIG. 8 only extends out to ±tₘₐₓ/2 for object 2. Reconstructed image 904 shows object 1. Although its width is fully resolved, it has aliasing artifacts 906 associated with it, which may be caused by the part of signal 802 in FIG. 8 that extends beyond ±tₘₐₓ/2 for object 1, which is ±7.5 seconds.

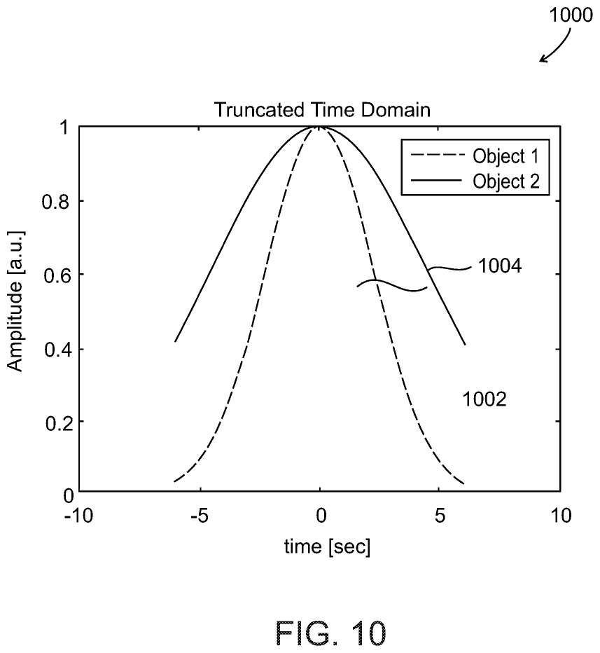

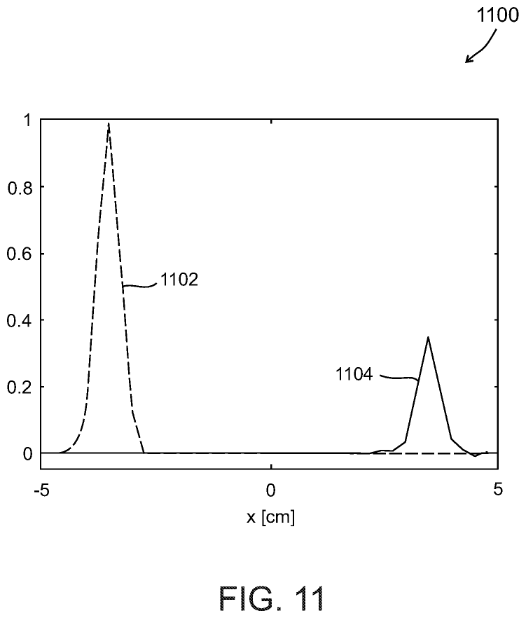

FIG. 10 shows a schematic plot 1000 of the MRI signals for object 1 and object 2, with the acquisition time truncated at ±6 seconds for both objects. Signal 1002 is for object 1, and signal 1004 is for object 2. FIG. 11 shows a schematic plot 1100 of a reconstructed image of object 1, curve 1102, and object 2, curve 1104, using the truncated MRI signals 1002 and 1004. In the reconstructed image, object 1 no longer shows aliasing artifacts, because the acquisition time of MRI signal 1002 has been limited to ±tₘₐₓ for object 1, and it has nearly full resolution. But object 2 shows a loss of resolution with the peak of m(x) greatly reduced, because the MRI signal has been limited to an acquisition time much shorter than ±tₘₐₓ/2 for object 2. No single value of the acquisition time produces good reconstructed images for both object 1, at a high field gradient G, and object 2, with a low field gradient G. A better image might be produced if a different acquisition time were used for object 1 and object 2, based on their local field gradients. This is known as an adaptive acquisition time.

Ideally, one would like to implement adaptive acquisition time by truncating the MRI signal coming from each point x in the FOV to an acquisition time window of duration π/δₓγG(x), which is the general expression for tₘₐₓ noted above. Optionally, the desired resolution δₓ could also be a function of x, if different parts of the FOV should have different resolution. However, it is generally not possible to tell, for a given projection, which parts of the signal come from which locations x. But it is possible to distinguish components of the signal with different frequencies ω, that come from locations x with different values of B(x), since only locations with B=ω/γ contribute to the signal component with frequency ω. For typical spatially encoding magnetic fields B(x), there is a significant correlation between B(x) and G(x), with higher field regions tending to also have higher field gradient. In addition, it is possible to partially distinguish between components of the signal that come from different locations, even locations with the same B, if multiple receiver antennas are used, and the signals from different antennas are recorded separately, because different receiver antennas generally have different sensitivities c(x). So optionally, adaptive acquisition time can be implemented by assigning to the frequency component for each frequency band ωₗ, for each receiver antenna, a value of acquisition time t_{acq}=π/δₓγGₗ based on a characteristic field gradient Gₗ for those locations x in the FOV for which γB(x) is in the frequency band ωₗ, and optionally characteristic in some sense of those parts of the FOV that predominantly contribute to the signal from that receiver antenna. It should be understood that even though the characteristic gradient Gₗ is only labeled by the subscript l, indicating which frequency band ωₗ it is used for, different characteristic gradients are also optionally associated with different receiver antenna components of the signal, and in that case Gₗ is also implicitly labeled with an index indicating which receiver antenna component it is used for.

A study by the inventors, described in the Examples section, has shown that this method of adaptive acquisition time, but only using a single receiver antenna, can produce substantial improvement in reconstructed image quality, compared to the best images that can be reconstructed using only a single value of acquisition time. The inventors have found that part of the improvement in reconstructed image quality, associated with adaptive acquisition time, is due to reduced sensitivity to noise. When the acquisition time is reduced for all parts of the FOV, in order to avoid aliasing in regions of highest field gradient, this not only can cause a loss of resolution in the lower gradient parts of the FOV, but can also cause an increased sensitivity to noise, due to the encoding matrix being ill conditioned. Using adaptive acquisition time can help to avoid that problem, because the longer acquisition times can provide some redundancy which makes the encoding matrix better conditioned.

Optionally, if the highest priority is to avoid any aliasing, even at the cost of lower resolution at some locations, then Gₗ may be set equal to the highest field gradient at any point x for which γB(x) is in the frequency band ωₗ. Alternatively, if avoiding aliasing and not losing resolution are both important, then Gₗ may be set equal to an average value, for example the mean or median value, of the field gradient at any point x for which γB(x) is in the frequency band ωₗ. Alternatively, a certain percentile of the field gradients for those locations x is used, for example an 80^{th} percentile if it is desired to put more emphasis on avoiding aliasing, or a 20^{th} percentile if it is desired to put more emphasis on not losing any resolution. Using a fixed percentile has the potential advantage, for example, of not using the maximum gradient if the maximum gradient is much higher than typical values of the gradient and is only found in a small part of the FOV where having some aliasing might not matter very much.

Optionally, if an average or percentile value of the field gradient is used, then, for a component associated with one receiver antenna, the average or percentile is weighted toward those values of x for which the sensitivity c(x) for that receiver antenna is higher, for example weighted by c, or by some monotonic function of c, for example the square of c. Optionally, this weighting by c, or by a monotonic function of c, for each antenna, is normalized by a sum of values of c, or values of the monotonic function of c, for all of the antennas at that x. Optionally, for a component associated with one receiver antenna, the rule for selecting the characteristic gradient Gₗ excludes locations x for which c(x) for that receiver antenna is considered negligibly small because it is below some specified value, and/or because it is much smaller than c(x) for other receiver antennas.

Optionally, the time window Wₗ(t) for acquiring the signal in a given frequency band l, centered at frequency ωₗ, has a constant value over a time interval of a width equal to the adaptive acquisition time for that frequency band, and is zero outside that time interval. This might be true, for example, if the adaptive acquisition time is implemented in hardware, with a receiver, sensitive only to that frequency band, records data only within that time interval Alternatively, Wₗ (t) does not cut off sharply at the beginning and end of the acquisition time interval, but goes more gradually to zero at the beginning and end of the acquisition time interval, which has the potential advantage that it may help to reducing ringing artifacts in the reconstructed image. In this case, the effective acquisition time for that frequency band may be considered to be, not the total width of the time interval over which Wₗ(t) is non-zero, but twice the standard deviation of Wₗ(t). Optionally, that definition of the effective acquisition time is used even if Wₗ(t) cuts off sharply.

This adaptive acquisition method can be implemented directly at the receiver level, where different frequency bands are acquired separately for different periods of time, or digitally as an additional processing step. For the latter implementation the signal s(t) is recorded for a duration of at least tₘₐₓ, which is used here to mean the longest of the acquisition times t_{acq}(ωₗ) for any frequency band ωₗ. It is then Fourier transformed to find generalized projections P_{θ} in the frequency domain

$${P}_{θ} ( {ω}_{l} ) = {\int }_{- {t}_{max / 2}}^{{t}_{max / 2}} s ( t ) W ( t ) exp ( i {ω}_{l} t ) dt \qquad ( 4 )$$

The window function W(t) is used here to reduce spectral leakage. Finally, we project P_{θ} back to the time domain by multiplying each coefficient P_{θ}(ωₗ) by the appropriate complex exponential function of effective duration t_{acq}(ωₗ), given by

$$\\ \begin{matrix}{F}_{l}^{w} ( t ) = exp ( - i {ω}_{l} t ) W ( \frac{{G}_{l}}{{G}_{min}} t ) & ( 5 )\end{matrix}$$

where each window

$$\\ {W}_{l} ( t ) = W ( \frac{{G}_{l}}{{G}_{min}} t )$$

is a scaled version of the window W(t) and the scaling factor Gₗ/Gₘᵢₙ regulates its time duration. The division by W(t)is meant to compensate for the multiplication by the same function in eq (4). The anti-aliasing-filtered version of the signal in the time domain takes the form

$$\\ \begin{matrix}{s}_{aa} ( t ) = \frac{1}{w ( t )} {\sum }_{l} {P}_{θ} ( {ω}_{l} ) {F}_{l}^{w} ( t ) & ( 6 )\end{matrix}$$

Optionally the Fourier transform of the windows function Wₗ(t). has an effective support small enough so that the k-space approximation holds within it, which has the potential advantage of making the anti-aliasing process more effective.

If Eq. (1) is used to express the unfiltered signal s(t) in terms of the image m(x), and Eqs. (4), (5), and (6) are used to expressed the filtered signal sₐₐ(t) in terms of s(t), then the filtered signal can be expressed in terms of m(x). Similarly, the filtered signal can also be expressed in the frequency domain:

$$\\ \begin{matrix}{P}_{θ , aa} ( ω ) = \int {{B}_{θ} ( x )}^{2} cos ( {ϕ}_{θ} ( x ) ) c ( x ) m ( x ) {\sum }_{l = 0}^{{N}_{t} - 1} \hat{W} ( {ω}_{l} - γ {B}_{θ} ( x ) ) ( ω - {ω}_{l} ) dx & ( 7 )\end{matrix}$$

where [symbol] is the inverese Fourier transform of Fₗ^{w}(t) and is given by the convolution of

$$\\ {\hat{W}}_{l} ( ω - {ω}_{l} ) = \hat{W} ( \frac{{G}_{min}}{{G}_{l}} ( ω - {ω}_{l} ) )$$

and the convolutional inverse of Ŵ(ω). Since Wₗ has a small support, the value of P_{θ,aa} at any frequency is the weighted sum of a small number of Fourier coefficients P_{θ}(ωₗ).

To reconstruct an image from the filtered signal, either in the time domain or the frequency domain, the image is expressed as a vector m whose components are m(x) in each pixel or voxel, and the filtered signal is expressed as a vector whose components are the value of the filtered signal at each of a discrete set of sampling times, or the value of filtered signal at each frequency band ωₗ, for each projection, for each receiver antenna if their signals are saved separately. All of these components are stacked into one big vector. An encoding matrix E transforms the image vector into the signal vector. Reconstructing the image vector from the signal vector involves finding an inverse or pseudo-inverse of the encoding matrix E, and optionally using a regularization term.

### Method of Reconstructing the Image

Flowchart 600 in FIG. 6 shows details of how the image is reconstructed from the MRI signal, according to an exemplary embodiment of the invention, where an iterative method is used to reconstruct the image. There are other embodiments where iteration is not used, but where, for example, an image is found directly, without the need for iteration. Tikhonov regularization, for example, can give a direct analytic solution for the reconstructed image, given by Eq. (9) below.

At 602, a received filtered MRI signal is provided. At 604, an encoding matrix is provided that transforms an image vector into the signal vector that it would produce. At 606, an inverse or pseudo-inverse of the encoding matrix is found. At 608, the filtered signal vector is multiplied by the inverse or pseudo-inverse to find an initial trial image vector.

At 610, the trial image vector is multiplied by the encoding matrix to find an expected filtered signal vector for the trial image. At 612, a positive definite measure is found of the difference between the expected filtered signal vector for the trial image, and the received filtered signal vector.

At 614, the positive definite measure of the difference is added to a regularization term that depends on the trial image vector. The regularization term is large if the trial image vector is noisy, or is otherwise thought not to represent a physically reasonable image.

At 616 it is determined whether the sum of the positive definite difference and the regularization term is low enough so that the reconstructed image can be considered converged. If not, then at 618 a new trial image vector is found, that would be expected to minimize the sum of the positive definite measure and the regularization term, making a fit to the present value of the sum and present trial image vector, and the value of the sum and the trial image vector at one or more previous iterations. The flowchart then returns to 610, using the new trial image, for the next iteration. Some iterative methods that may be useful for this purpose are given, for example, by Beck and Teboulle, cited above.

If the reconstructed image is considered to be converged, then the reconstructed image is set equal to the trial image at 620, and the method ends at 622.

It should be understood that finding the minimum of the positive definite measure of the difference, with or without a regularization term added to it, does not, in general, mean finding an absolute minimum, but finding an approximate minimum to a chosen precision. For example, if the minimum is found using an iterative search method, then the search may end when the quantity being minimized changes by less than an amount corresponding to the chosen precision from one iteration to the next. The chosen precision is chosen, for example, based on the best precision possible given a known noise level of the filtered received signal.

### Regularization Methods

As noted previously, when there is a large ratio between the minimum and average field gradient in the FOV, the number of time samples required for exact reconstruction can be much larger than the number of pixels Nₓ across the FOV, and the number N_{γ} of projections, such as rigid rotations of the magnetic field, can be quite large as well, leading to heavier computation and longer scan times.

Therefore, in practical applications the acquisition time is shortened and the number of rotations reduced, causing the inverse or pseudo-inverse of the encoding matrix for the filtered signal to be ill-conditioned and the resulting estimate \hat{m} of the reconstructed image to be very sensitive to noise and suffering from undersampling artifacts.

To mitigate these shortcomings and achieve a more stable reconstruction scheme, prior knowledge can be added to the optimization problem, in the form of regularization terms, as described for example by Yonina C Eldar. Sampling theory: Beyond bandlimited systems. Cambridge University Press, 2015. When a regularization term is used, the reconstructed image is the image \hat{m} for which the positive definite measure of the difference between the received signal and the expected signal for that image, plus the regularization term, is minimized:

$$\\ \begin{matrix}\hat{m} = {arg min}_{m} {\underbrace{{‖ s - Em ‖}_{2}^{2}}}_{data consistency} + λ {\underbrace{f ( m )}}_{prior} \{ \begin{matrix} \\ \end{matrix} \} & ( 8 )\end{matrix}$$

The first term, the positive definite measure of the difference, is called the data consistency term because it evaluates how closely the reconstructed image vector would produce a signal that approximates the received measurement vector s. The second term in Eq. (8), labeled “prior”, incorporates prior knowledge of what the image is expected to look like, for example in medical MRI it is expected that the image will often consist of large fairly uniform areas with sharp boundaries between them λ is a constant that balances between the data consistency term and the prior.

In tests described in the Examples section, the inventors implemented two types of regularization, Tikhonov and lᵢ, where the latter is implemented in the context of Compressed Sensing (CS).

### Tikhonov Regularization

Tikhonov regularization imposes a constraint on the l₂ norm of the solution f(m)=∥Γm∥₂². When Γ is a high pass operator, it penalizes strong edge, while another common choice implemented in this work is Γ=I, with I being the identity matrix, which gives the minimum norm solution

$$\\ \begin{matrix}\hat{m} = {arg min}_{m} \{ {‖ s - Em ‖}_{2}^{2} + {λ}_{Tikh} {‖ m ‖}_{2}^{2} \} = {( {E}^{H} E + {λ}_{Tikh} I )}^{- 1} {E}^{H} s & ( 9 )\end{matrix}$$

### Compressed Sensing

Natural images are often compressible, which means that under an appropriate sparsifying transform, a small subset of their coefficients account for most of the energy of the signal while the rest are negligible.

This property of natural images is leveraged by sparse recovery algorithms, that allow to solve under-determined optimization problems by adding a sparsity constraint to the solution. The sparsity constraint can be expressed as the l₀ quasi-norm of the transform coefficients, indicating the number of non-zero elements, so that our MR image reconstruction problem can be re-written as

$$\\ \begin{matrix}\hat{m} = {arg min}_{m} {‖ Ψ m ‖}_{0} s . t . {‖ s - Em ‖}_{2}^{2} < ε & ( 10 )\end{matrix}$$

where Ψ is the sparsifying operator (in this case the Wavelet transform), and ε is some positive constant accounting for measurement noise. Since the l₀ pseudo-norm produces a non-convex minimization problem, the constraint on the coefficients can be relaxed by using the convex l₁ norm that guarantees the existence of a global minimum to the problem and can be solved in polynomial time

$$\\ \begin{matrix}\hat{m} = {arg min}_{m} \{ {‖ s - Em ‖}_{2}^{2} + {λ}_{cs} {‖ Ψ m ‖}_{1} \} & ( 11 )\end{matrix}$$

where ∥⋅∥₁ is the l₂ norm, which penalizes non-sparse solutions. The above equation has also a canonical form where the transform coefficients c are retrieved, instead of the image:

$$\\ \begin{matrix}\hat{c} = {arg min}_{c} \{ {‖ s - Γ c ‖}_{2}^{2} + {λ}_{cs} {‖ c ‖}_{1} \} & ( 12 )\end{matrix}$$

where Γ=EΨ* is called the sensing matrix and it describes the relation between the transform coefficients and the measured signal, while Ψ* is the adjoint of the (orthogonal) sparsifying transform.

In compressive sensing, the maximal undersampling rate that still allows for a faithful reconstruction of the image depends both on the degree of sparsity of the image and the coherence of the sensing matrix. The coherence is defined as the largest absolute normalized inner product between any two columns gᵢ and gⱼ of Γ

$$\\ \begin{matrix}μ ( Γ ) = {min}_{1 \leq i < j \leq {N}_{v}} \frac{\text{\textbar <} {g}_{i} , {g}_{j} \text{>\textbar}}{{‖ {g}_{i} ‖}_{2} {‖ {g}_{j} ‖}_{2}} & ( 13 )\end{matrix}$$

Intuitively, a large coherence indicates that two Wavelet coefficients have very similar representations in the time domain and therefore, a small perturbation of the signal can trick the algorithm into choosing the wrong coefficient. For example, consider a sparse image (Ψ=I) and an encoding matrix in the Fourier basis (Discrete Fourier Transform), as in conventional MRI. In such a case, uniformly undersampling E by increasing Δₖ results in aliasing in the image because pairs of pixels have exactly the same encoding function. In this case the coherence is 1 and the image cannot be recovered. Conversely, When the DFT matrix is randomly undersampled, the coherence is lower and the image can be reconstructed if it is sparse enough. The sparsity requirement given a specific coherence μ(Γ) is that the number S of non-zero coefficients in the transform domain of the image is at most

$$\\ \begin{matrix}S < \frac{1}{2} ( 1 + \frac{1}{μ ( Γ )} ) & ( 14 )\end{matrix}$$

Designing undersampling patterns with low coherence may make it possible to reconstruct the image from fewer measurements.

With compressive sensing, the number of projections is optionally reduced by at least a factor of 2 from Nᵣ, or at least a factor 5, or at least a factor 10, with relatively little reduction in image quality. In the case of a three-dimensional FOV, the different projections of the spatially encoding magnetic field are optionally produced by rigidly rotating the magnetic field source around two different axes, for example two axes that oriented at approximately right angles to each other, and, without using compressive sensing, a total of Nᵣ² different projections may be required to produce a full resolution image without image artifacts. With compressive sensing, using a regularization term of this form, the number of projections is optionally reduced by a factor of 2, or 5, or 10, or 25, or 100, from Nᵣ², with relatively little reduction in image quality.

### Simulation Results

In modern medicine, Magnetic Resonance Imaging (MRI) is a fundamental diagnostic tool and is particularly suited for acquiring high resolution images of soft tissues. Despite their unique features, MRI scanners are sometimes not widely accessible due to very high production and maintenance costs. Moreover, due to their strong magnetic field, MRI scanners are often confined to dedicated rooms in large medical centers that abide by stringent safety requirements. These limitations may preclude MRI from daily use in small clinics or in field medicine. A goal of this theoretical study was to demonstrate a proof of concept of a scaled-down MRI scanner that does not use a high and a homogeneous magnetic field and can potentially be portable.

Our framework, in an exemplary embodiment of the invention, is based on a weak and highly inhomogeneous magnetic field, that does not require superconductors and refrigeration systems. Moreover, the inhomogeneity of the field is exploited for spatial encoding, so that no pulsed-gradient coils are needed. The proposed method imposes very loose constraints on the magnetic field shape and is therefore applicable to a wide variety of magnets, including open magnets, and is potentially cheap to implement.

We focused in this work on the encoding and reconstruction process of a two dimensional object with the following 4 outcomes: (a) Image encoding process that doesn't require gradient coils. (b) Development of mathematical tools for analyzing the resolution and noise propagation of different reconstruction methods, for a given magnetic field. (c) A sampling scheme and reconstruction algorithm that account for the variations in the field's intensity in order to reconstruct the imaged object with nearly uniform resolution. The proposed algorithm was compared with the leading algorithm in the literature, both theoretically and through simulations, and it achieved better resolution and improvement in image signal to noise ratio (SNR) by 3 to 8 dB, depending on the SNR of the measurements. (d) Application of the Compressed Sensing framework allowed to reduce scan time from 50 to 10 minutes.

The image reconstruction methods involve storing an encoding matrix of size NₜN_{γ}xNᵥ, which for a 256×256 image, 973 rotations and 738 single-precision complex time samples (see next section), would require roughly 350 GB of RAM. This requirement can be relaxed by iteratively building the WLS (Weighted Least Squares fit) solution, so that equation (38), for instance, takes the following form

$$\\ \begin{matrix}\hat{m} = {arg min}_{m} {\sum }_{θ ∈ Θ} {‖ {Λ}_{θ}^{- \frac{1}{2}} ( {s}^{θ} - {E}_{t}^{θ} m ) ‖}_{2}^{2} := {A}^{- 1} d & ( 15 )\end{matrix}$$

Here Nᵥ is the number of voxels or pixels in the FOV, Nᵣ is the number of projections, and Nₜ is the number of time samples taken for the signal at each projection. A= [symbol] Eₜ^{H,θ}Λ_{θ}⁻¹ Eᵣ^{θ} is of size NᵥxNᵥ, and d= [symbol] Eₜ^{H,θ}Λ_{θ}⁻¹s^{θ} is a Nᵥx1 vector. Here θ is a label for the projection, for example θ could be the angle of orientation of the spatially encoding magnetic field if the different projections are rigid rotations of the spatially encoding magnetic field of the first projection around one axis. Λ_{θ} is the non-white noise covariance matrix for the signal s_{θ}(t) for projection θ, in the time domain, which is used for finding a WLS fit for the reconstructed image, taking into account the non-white nature of the noise. In general, the noise in an MRI signal may not be white noise, but may increase linearly with frequency ω, because the voltage in a receiver coil, for a given RF magnetic field, may be proportional to ω. This approach allows to iterate over θ and store after each step the cumulative sums that add up to A and d which amount to 24 GB and 384 KB respectively.

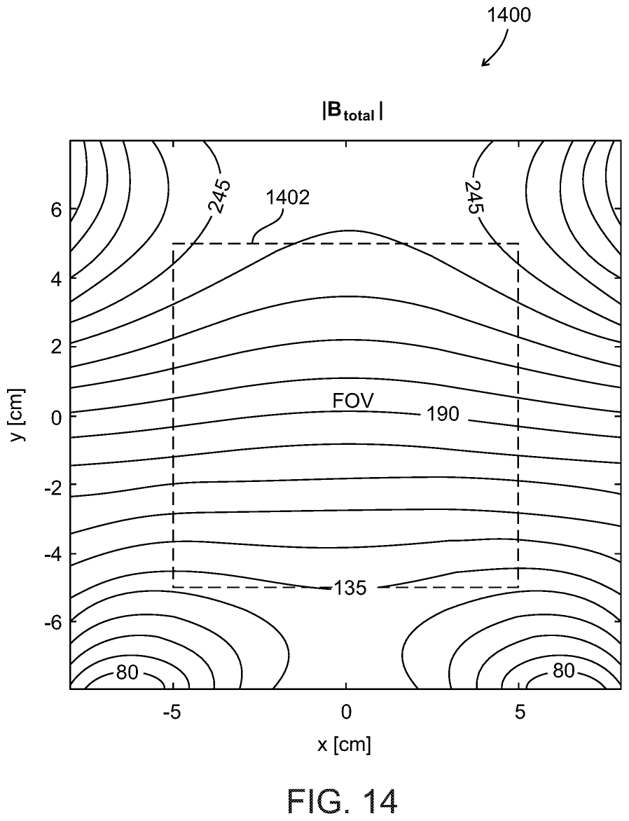

Furthermore, the imaged object usually occupies only part of the FOV (FIG. 14), while the surrounding doesn't contribute any signal. If a region of interest (ROI) could be defined inside the FOV, for example by using markers on the circumference of the object, then only the voxels inside the ROI would be taken into account when defining the encoding matrix. This subterfuge would reduce not only Nᵥ, but also Nₜ and N_{γ} because the stimulated bandwidth BW would be smaller.

While the above adaptations apply to all the reconstruction methods, a further pre-processing step is required for compressed sensing, namely normalizing the encoding matrix. For simplicity of notation, we define the following encoding matrix E=Λ^{-1/2}Eₜ, so that A=E^{H}E, and we decompose E to the product E=\tilde{E}Q, where \tilde{E} is the normalized encoding matrix where each column has unit norm, and Q is a NᵥxNᵥ diagonal matrix with elements qᵢᵢ=\sqrt{gᵢ^{H}eᵢ}, where gᵢ is the i^{th} column of E. Under this new representation, the reconstruction problem can be rewritten as

$$\\ \begin{matrix}{\hat{m}}_{cs} = {arg min}_{\tilde{m}} {‖ s - \tilde{E} \tilde{m} ‖}_{2}^{2} + {λ}_{cs} {‖ \tilde{Ψ} \tilde{m} ‖}_{1} & ( 16 )\end{matrix}$$

where \tilde{m}=Qm and \tilde{Ψ}=ΨQ⁻¹ and the voxel values are retrieved as \tilde{m}=Q⁻¹\tilde{m}_{CS}.

For the Wavelet operator Ψ, the 4 levels Daubechies discrete Wavelet Transform (DWT) implementation by Lustig

(Lustig M, Donoho D L, Santos J M, Pauly J M (2008) Compressed sensing MRI. IEEE Signal Process Mag 25(8):72-82. doi:10.1109/MSP.2007.914728; see also Daubechie I (1992) Ten lectures on wavelets. In: CBMS-NSF conference series in applied mathematics. www(dot)dx.doi.org/10.1137/1.9781611970104) was adopted and the FISTA algorithm (P. C. Lauterbur, “Image formation by induced local interactions: Examples employing nuclear magnetic resonance,” Nature 242(5394):190-191, Mar. 1973) was used in order to solve the above optimization problem.

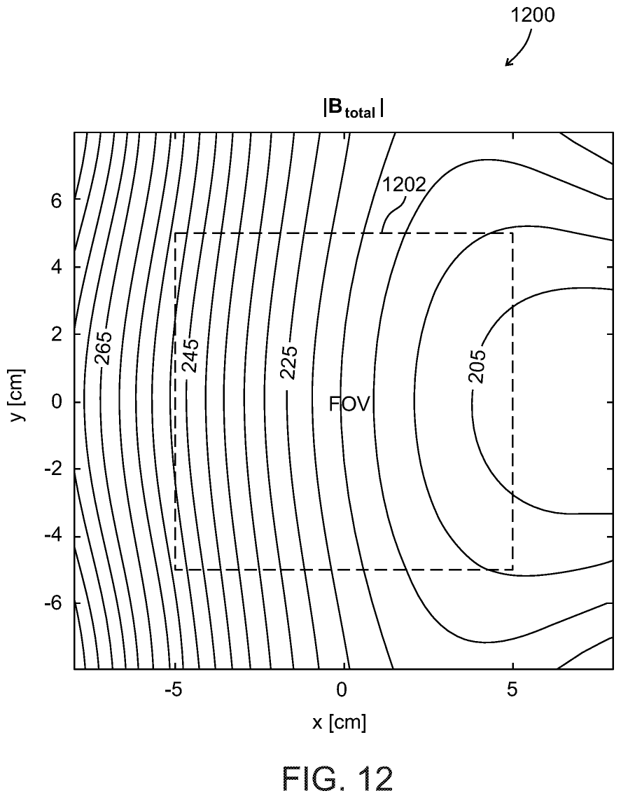

Simulations of image reconstruction were done for two magnetic field configurations using permanent magnets: a U-shape magnet, and a cap magnet. In both cases, the different projections were obtained by rigid rotation of the magnetic field source around one axis, perpendicular to the plane of the FOV, in the center of the FOV. The U-shaped magnet and the location of its FOV was described previously in FIG. 4. FIG. 12 shows a contour plot for the magnitude B of the magnetic field, which is mostly in the z direction, as a function of coordinates x and y within FOV 1202. The contours are all 4 mT apart, and the field values for some of the contours, at 205 mT, 225 mT, 245 mT, and 265 mT, are shown as labels on those contours. Both the field magnitude B, and the gradient of B, decrease going from left to right in the FOV, and the field magnitude and its gradient are fairly well correlated, with relatively little variation in the magnitude of the gradient along a given contour of constant B.

A cap magnet configuration 1300 is shown in FIG. 13. The cap magnet is made of a hemispherical layer of permanent magnet material 1302, magnetized with a north pole on the inner surface and a south pole on the outer surface. A hemispherical layer of iron 1304 on the outer surface of the magnet, and a hemispherical layer of iron 1306 on the inner surface of the magnet, both have uniform thickness and thick enough to avoid saturating. The hemispheres are axially symmetric around the y-axis. The magnet produces an external magnetic field 1308, predominantly in the y-direction, and the layers of iron keep the magnetic field nearly perpendicular to the surface of the magnet at its inner and outer surfaces. A field of view 1310, 10 cm×10 cm, is located in the x-y plane at z=0, and is centered on the y-axis, at the value of y where the gradient (mostly in the y-direction) in the magnitude B of the magnetic field is greatest. A motor or actuator, for example similar to motor 416 in FIG. 4, causes magnetic cap configuration 1300 to undergo a rotation 1312 around the z-axis, located for example at the center of FOV 1310, to produce different projections of the spatially encoding magnetic field in FOV 1310, which remains fixed in orientation when the magnetic cap rotates. A full rotation of 180 degrees is used, for example, with the different projections spaced closely enough in angle to provide a good quality reconstructed image, with or without compressed sensing.

It should be understood that magnetic cap 1300 is very different from the magnetic cap described by McDaniel et al, cited above. McDaniel's cap has a magnetic field predominantly perpendicular to the vertical axis of a human subject's body, when the cap is placed on top of the subect's head. The gradient in field magnitude is predominantly in the vertical direction, perpendicular to the field direction. The cap does not rotate to produce different projections of the magnetic field, but has gradient coils to produce different projections of the magnetic field. In magnetic cap 1300, the field is predominantly in the direction of the axis of rotational symmetry of the cap, and the gradient in field magnitude is predominantly in the same direction. To produce different projections of the magnetic field, the cap rotates not around its axis of rotational symmetry, which would not produce any change in the magnetic field, but around an axis perpendicular to the axis of rotational symmetry.

FIG. 14 shows a contour plot 1400 of the contours of magnetic field magnitude B for the cap magnet, in FOV 1402, for the cap magnet at one possible orientation with respect to the FOV, for example the orientation used for the first projection. The contours are 11 mT apart, and the field values for some of the contours, at 80 mT, 135 mT, 190 mT, and 245 mT, are shown as labels for those contours. The total range of magnitude of field gradient G in FOV 1402, for the cap magnet, is somewhat less than the total range of the magnitude of field gradient in FOV 1202 for the U-shaped magnet. But the variation in field gradient along each contour of B, in the FOV, is somewhat greater on average for the cap magnet than for the U-shaped magnet. Another different between the U-shaped magnet configuration and the cap magnet configuration is that the magnetic field is primarily in the z-direction, perpendicular to the plane of the FOV, in the U-shaped magnet configuration, while the magnetic field is primarily in the y-direction, in the plane of the FOV, in the cap magnet configuration. Also, for the U-shaped magnet the field gradient is predominantly perpendicular to the direction of the field, with the field lines curvature balancing the field gradient, while for the cap magnet the field gradient is predominantly parallel to the direction of the field lines, which fan out. The good results obtained for image reconstruction, using adaptive acquisition time, for both of these very different magnet configurations, is an indication of the general applicability of the method to a wide variety of different magnet configurations.

The simulations were performed with Matlab on an ASUS G752VY laptop with a 4 Cores Intel(R) Core(™) i7-6820HK CPU @ 2.70 GHz and 32 GB of RAM. In all the experiments the 256×256 pixels Shepp-Logan phantom with uniform pixel size Δₓ=1 mm was encoded and then reconstructed with different algorithms and parameters. First, the column-stacked image is projected into the time domain by the encoding matrix E_{θ}, corresponding to each rotation of the magnet. Then, each acquired time signal undergoes anti-aliasing filtering, according to the algorithm being tested (constant acquisition time method, or adaptive acquisition time method). Then the image is reconstructed using either Tikhonov regularization or compressed sensing (CS). and finally, the reconstruction error is measured in dB as the norm of the differences between the encoded and the reconstructed image, divided by the norm of the encoded image:

$$\\ \begin{matrix}image SNR = 20 log ( \frac{{‖ \hat{m} - m ‖}_{2}}{{‖ m ‖}_{2}} ) & ( 17 )\end{matrix}$$

where m is the original image and \tilde{m} is the reconstructed image.

The constant and the adaptive acquisition time methods were compared at different SNR levels to evaluate the achievable resolution of each method and its performance in the presence of noise. The noise samples were drawn from a Gaussian distribution with a PSD that increased linearly with the frequency of the RF signal, and its variance was set at 12, 20, 26, 32 and 40 dB below the power of the time signal. For the U-shaped magnet the magnet was rotated 181 degrees at increments of Δ_{θ}=0.13 5° (eq. 31), for a total of N_{θ}=343 rotation angles. the sampling rate was set to fₛ=1.2 BW=339:3 MHz and Nₜ=1019 samples were collected for the adaptive acquisition time method, based on the smallest field gradient in the FOV, as opposed to Nₜ=170 for the constant acquistion time method, based on the largest gradient in the FOV. To evaluate the resolution obtained with the two methods, the point spread function (PSF) was computed at (x=0, y=0)and(x=0, y=−4) cm for both methods by encoding and reconstructing and image composed of only one non-zero pixel at the chosen location.

The cap magnet was rotated by 181 degrees as well, at increments of Δ_{θ}0.186°, for a total of N_{θ}=973 rotation angles. The sampling rate in this setting was fₛ=4.8748 MHz and Nₜ=738 for the adaptive acquisition time method, while Nₜ=220 for the constant acquisition time method.

For the simulations, the coil sensitivity c(x) is assumed to be constant, except for some of the simulations using the cap magnet, as will be described. Moreover, both the adaptive acquisition time method and the constant acquisition time method used some regularization because the encoding matrix was close to singular, so Tikhonov regularization was applied to both methods, and λ_{Tikh} was chosen as the maximum of 0.035 and the noise variance Λ.

The adaptive acquisition time method was then tested when the number of rotations was reduced to N_{θ}=195 and N_{θ}=97, corresponding to compression factors of R=5 and R=10 respectively and the image was reconstructed both with Tikhonov regularization and CS with λ_{cs}=10⁻⁴ for 30 dB SNR or higher and λ_{cs}=10⁻² below that to increase denoising. The undersampling consisted of increasing Δ_{θ} by a factor of R, and still rotating the magnet at constant increments. Because of the non-linear profile of the magnetic field, the constant increments in θ don't correspond to a uniformly undersampled k-space. Nonetheless, an attempt was made to randomly select the subset of rotation angles in order to reduce the coherence of the matrix Γ, but no significant improvement was achieved with the limited number of configurations tested. Instead, the center of rotation was randomly displaced with a uniform distribution U(−0.5 cm, 0.5 cm) in each direction and this successfully reduced undersampling artifacts.

The reconstruction methods generally rely on the assumption that the magnetic field intensity and direction is known throughout the FOV. In practice, B₀ is measured with some error on a set of locations within the FOV, and is then fitted to spherical harmonics base functions in order to interpolate its value at each voxel center. In order to evaluate the robustness of the proposed algorithm to B₀ mapping errors, two more simulations were run, where two different types of calibration error were added to the magnetic field maps, prior to image reconstruction: The first error consisted of a constant error of 100 μT, added to each component of the field, while the second type of error was a uniformly distributed random error in the range [−50,50] μT that was drawn separately for each voxel center and rotated together with the magnetic field. These constant and white noises can, for example, cover the extreme cases where the calibration error has either full correlation or is completely uncorrelated.

The relative amplitude of the errors in the reconstructed image in the simulations, with respect to the original m(x), referred to as the SNR of the reconstructed image, was evaluated as a function of the SNR of the MRI signal, which had noise introduced added to it in the simulations. When there was a high level of noise introduced into the MRI signal, i.e. when the SNR of the MRI signal was low, the error level in the reconstructed image was dominated by the noise in the signal, and the SNR of the reconstructed image was proportional to the SNR of the signal, i.e. the SNR of the image and the SNR of the signal had a constant difference in dB. When the relative noise level introduced into the signal was sufficiently low, i.e. when there was a sufficiently high SNR for the signal, then the error level in the reconstructed image was dominated by artifacts of the reconstruction, such as aliasing or ringing artifacts, or blurring of detail due to limited acquisition time, rather than by the noise in the signal, and the SNR of the reconstructed image was nearly independent of the SNR of the signal. In both the low noise and high noise limits, and everything in between, the SNR of the reconstructed image was higher, for a given SNR of the signal, when the adaptive acquisition time method was used, than when the constant acquisition time method was used.

For example, when the full set of projections were used, without compressive sensing, and Tikhonov regularization was used, with the U-shaped magnet, the SNR of the image was 11.5 dB for the constant acquisition time method, in the limit of high SNR for the signal, but was 36.5 dB for the adaptive acquisition time method, in the limit of high SNR for the signal. With the cap magnet, the SNR of the image was 23 dB for constant acquisition time method, in the limit of high SNR for the signal, greater than about 32 dB, and was 31 dB for the adaptive acquisition time method, in the limit of high SNR for the signal, or 8 dB higher than with the constant acquisition time method. In the limit of high noise, i.e. signal SNR at a given value well below about 26 dB, the SNR of the reconstructed image was about 4 dB higher for the adapative acquisition time method, than for the constant time acquisition method.

The resolution of the image for the U-shaped magnet, using the constant acquisition time and adaptive acquisition time methods, was compared, by calculating the Point Spread Function (PSF) at different locations in the FOV, by using an original image m(x) that was non-zero only in one pixel at a time. It was found that the resolution was higher everywhere with the adaptive acquisition time method, than with the constant acquisition time method, but the difference was especially great near the edge of the image. The resolution was nearly uniform throughout the FOV using the adaptive acquisition time method, but was higher near the center of the FOV using the constant acquisition time method, because the field gradients were stronger there.

The reconstructed image SNR was also measured using under-sampled signals, with the number of projections reduced by a factor of R=5, or R=10, using Tikhanov regularization, and using compressive sensing. Strong under-sampling artifacts are seen in the images using Tikhanov regularization, with R=5 and even more with R=10, which decreases the SNR of the reconstructed image. With compressive sensing, the SNR of the reconstructed image is not greatly reduced at R=5 and R=10. The main effect on the image of under-sampling is to produce noise near the edge of the image, but the resolution is hardly affected even at the edge. Randomizing the location of the axis of rotation of the spatially encoding magnetic field could improve the SNR of the reconstructed image by about 3 dB.

When the image was reconstructed with corrupted magnetic field maps, image quality degraded by 2 dB in the case of a random mapping error and by 6 dB with a constant error. No loss of resolution can be observed in the reconstructed images and the error manifested as a high frequency noise, spread throughout the image. As the SNR of the signals approached 20 dB and below, the SNR of reconstructed image converged, indicating that the signal noise became the main source of error.

When an inhomogeneous coil sensitivity map was used, the resulting image was almost indistinguishable from the one obtained with a homogeneous map. Image SNR was slightly higher at 31:6 dB, as opposed to 31 dB and the Gibbs effect detectable in the details image from the homogeneous map was reduced in the inhomogenous case.

The simulation results show that the adaptive-time method outperforms the constant-time method, both in terms of resolution, and robustness to noise. The constant-time method truncates the signal from pixels with weaker gradients, thus acquiring only the lower spatial frequencies in that portion of the FOV, and leaving out a portion of the energy of the signal. This approach may have a blurring effect on the image and may amplify noise because of the small condition number of the encoding matrix. Conversely, the adaptive acquisition time method may allow collecting also the higher spatial frequencies, while shortening the acquisition time for those values of B, and those receiver antennas, with stronger gradients, to avoid collecting unnecessarily noisy samples.

With the adaptive time method, assuming a repetition time (TR) of 3 seconds between rotations, a complete 2D scan with 973 rotation angles would take about 50 minutes. The application of the compressed sensing framework allows to reduce the acquisition time to 10 or even 5 minutes, by undersampling the number of rotations by a factor R of 5 and 10 times, respectively. For R=5 the undersampling manifests as a noise-like perturbation of the image, which is effectively corrected by the regularization implemented with CS. When R is increased to 10, undersampling artifacts start to appear in the image and although the regularization alleviates them, they still slightly affect image quality. Reducing the coherence of the encoding matrix by choosing a better subset of rotation angles and rotation center locations will likely reduce the artifacts further. Undersampling in the presence of noise has the additional drawback of reducing the number of spokes averaged at the center of the k-space and therefore, requiring more averages of the signal to make up for the loss of SNR.

The addition of an inhomogeneous coil sensitivity c(x) slightly reduced Gibbs effects and improved image SNR. This improvement is likely due to the modulating effect of c(x) that smoothed the high frequency components of the sharp edges of the phantom.

Although the reconstruction algorithm strongly relies on the knowledge of the magnetic field intensity, the addition of constant and random errors of about 1000 ppm to the B₀ map didn't affect significantly the quality of the image. Furthermore, as the SNR of the RF signal decreases, these mapping errors become negligible compared to the electromagnetic noise of the measurements.

As for the design of the magnet, the U-shaped magnet achieved higher SNR in the reconstructed images, both in the presence of noise and in its absence. The result in the noisy case is explained by the smaller RF bandwidth, while the better performance in the noise-free case is achieved because each constant B contour in the U-magnet subsumes a smaller range of gradient values compared to the cap magnet, and therefore less smearing takes place.

In this work we presented an acquisition scheme and a reconstruction algorithm for imaging without gradient coils and with a highly inhomogeneous magnetic field. The algorithm was compared with the leading algorithm in the literature, both theoretically and through simulations, and the presented method achieves nearly uniform resolution and is more robust to noise.

By implementing the compressed sensing framework, an image can be acquired in 10 minutes and reconstructed without any evident loss of precision, making the proposed method suitable for clinical use. The main downside of the proposed imaging method is the significantly lower SNR compared to a conventional 3T scanner, but the short signal duration allows to average numerous echoes within the same repetition time TR between excitation pulses. Moreover, the cap magnet used for the simulations has a completely open design to make it more versatile, but using a close configuration similar to a U-shaped magnet or even a Halbach magnet would greatly reduce the average gradient and along with it the bandwidth of the noise. Reducing the signal bandwidth would also allow to use a receiving coil with a smaller Q factor, increasing the SNR even further.

It is expected that during the life of a patent maturing from this application many relevant MRI pulse sequences will be developed, and many relevant regularization methods and iterative methods of reconstructing images with ill-conditioned encoding matrices will be developed and the scope of these term is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of scanning an object in field of view (FOV) by acquiring an MRI signal from the object in the FOV at a plurality of different projections of a spatially encoding magnetic field, using a plurality of receiving antennas, and reconstructing an MRI image of the object in the FOV comprising a plurality of voxels, the method comprising:
a) at each of the projections, acquiring the MRI signal from the receiving antennas, and recording data of the signal;
b) filtering the received signal with a signal filter, for at least some of the projections, by applying different time windows to at least some different components of the signal in different frequency bands, or received by different receiver antennas, or both, resulting in a filtered received signal vector whose components describe the filtered received signal as a function of time, at each projection, for one or more receiver antennas; and
c) reconstructing an image as a vector whose components describe a weighted or unweighted net magnetization at each voxel in the FOV, that would be expected to produce the filtered received signal vector.

2. A method according to claim 1, wherein recording the data comprises recording the data separately for at least some different receiver antennas or sets of receiver antennas, and filtering the received signal results in a filtered received signal vector whose components describe the filtered received signal as a function of time, at each projection, for each receiver antenna or set of receiver antennas for which the data is recorded separately.

3. A method according to claim 2, wherein at least some of the components of the signal received from different receiver antennas at a same or overlapping frequency band have different time windows.

4. A method according to claim 2, wherein the components of the filtered received signal vector for each projection, for each receiver antenna or set of receiver antennas for which the data is recorded separately, comprise values of the signal in different time intervals.

5. A method according to claim 2, wherein the components of the filtered received signal vector for each projection, for each receiver antenna or set of receiver antennas for which the data is recorded separately, comprise values of the signal in different frequency bands.

6. A method according to claim 1, wherein finding the reconstructed image vector comprises finding an image vector that minimizes a positive definite measure of a difference between the filtered received signal vector and a filtered signal vector that would be expected for the image vector.

7. A method according to claim 6, wherein the expected filtered signal vector for an image vector is an encoding matrix operating on the image vector, and finding the reconstructed image vector comprises multiplying the filtered received signal by an inverse or pseudo-inverse of the encoding matrix to find an image vector that would be expected to produce the filtered received signal vector.

8. A method according to claim 1, wherein finding the reconstructed image vector comprises finding an image vector that minimizes a sum of:
a) a positive definite measure of a difference between the filtered received signal vector and a filtered signal vector that would be expected for the image vector; and
b) a regularization term that depends on the image vector.

9. A method according to claim 8, wherein the positive definite measure of a difference between the filtered received signal vector and the expected filtered signal vector is a sum or weighted sum of squares of vector components of the difference.

10. A method according to claim 8, wherein the regularization term comprises a Tikhonov regularization term.

11. A method according to claim 8, wherein the regularization term comprises a constant multiplying a sum or weighted sum of absolute values of vector components of the image vector transformed by a matrix.

12. A method according to claim 11, wherein the matrix is a sparsifying transform.

13. A method according to claim 12, wherein the sparsifying transform is a wavelet transform.

14. A method according to claim 1, wherein the spatially encoding magnetic field is produced by a magnetic field source that rotates rigidly around at least one axis around the FOV, the different projections comprise a plurality of different rotation angles of the magnetic field source around the axis relative to the FOV, and the number of different rotation angles around the axis is lower by at least a factor of 2 compared to π/2 times a width of the FOV divided by a distance Δx that is resolved in the reconstructed image, times a ratio of an average magnetic field gradient in the FOV to an effective minimum magnetic field gradient in the FOV.

15. A method according to claim 1, wherein the spatially encoding magnetic field is produced by a magnetic field source comprising a plurality of elements at least some of which move and/or rotate relative to each other to produce the different projections, such that for at least two of the projections, the spatially encoding magnetic field for one of the projections is a non-rigid distortion of the spatially encoding magnetic field for the other projection.

16. A method according to claim 15, wherein the elements comprise one or more fixed elements that do not change their position or orientation between different projections, and one or more moving elements that do change their position or orientation between different projections.

17. A method according to claim 15, wherein the FOV includes at least part of a human subject's brain inside the subject's cranium, and for at least one projection the elements are located outside the subject's head, in a configuration with at least some of the elements sufficiently close to the subject's head that there is not room for the magnetic field source to rotate rigidly 180 degrees around the head.

18. A method according to claim 8, wherein finding the reconstructed image vector comprises searching for the image vector that minimizes the sum.

19. A method according to claim 6, wherein finding the reconstructed image vector comprises searching for the image vector that minimizes the positive definite measure of the difference.

20. A method according to claim 18, wherein searching for the image vector comprises using an iterative search method.

21. A method according to claim 1, wherein finding the image vector comprises calculating the image vector from an analytic expression for the image vector in terms of the filtered received signal vector.

22. A method according to claim 1, wherein values of the components of the filtered signal vector that would be expected for a given weighted or unweighted net magnetization as a function of voxel in the FOV, for each projection, are values that would be expected if, for every pair of different projections, the spatially encoding magnetic fields are rigid rotations of each other, relative to the FOV, over some angle around some axis.

23. A method according to claim 22, wherein said values are values that would be expected if, for all projections, the spatially encoding magnetic fields are rigid rotations of each other, relative to the FOV, over some angle around a single axis.

24. A method according to claim 1, wherein, for those projections for which the received signal is filtered, the time window has a standard deviation that is narrower, on average, for frequency component and receiver antenna combinations that have a characteristic magnetic field gradient magnitude that is higher than an average magnetic field gradient magnitude in the FOV, than for frequency component and receiver antenna combinations that have a characteristic magnetic field gradient magnitude that is lower than the average magnetic field gradient magnitude in the FOV.

25. A method according to claim 24, wherein the standard deviations of the time windows of the frequency component and receiver antenna combinations are linearly correlated with an inverse of the characteristic magnetic field gradient magnitudes of the frequency component and receiver antenna combinations, with a correlation greater than 0.5.

26. A method according to claim 25, wherein the voxels of the FOV have width of about a same value Δx, and a best linear fit of the standard deviation of the time window to the inverse of the characteristic magnetic field gradient magnitude Gₗ has the standard deviation of the time window between 1/(γGₗΔx) and 2.5/(γGₗΔx), where γ is the gyromagnetic ratio.

27. A method according to claim 1, wherein the MRI signal is received substantially only from a FOV only one voxel thick, with the voxels arranged in two dimensions in the FOV, and wherein, for each projection in at least one set of the projections that together contribute most of the MRI signal's energy, for a set of the voxels from which at least 90% of the MRI signal's energy is received, the spatially encoding magnetic field better distinguishes between components of the MRI signal received from each of those voxels and at least one adjacent voxel, than it distinguishes between components of the MRI signal received from different locations within the voxel.

28. A method according to claim 27, wherein the spatially encoding magnetic field is produced by a magnetic field source that rigidly rotates around the FOV on only one axis, and the different projections comprise different rotation angles of the magnetic field source around the axis with respect to the FOV.

29. A method according to claim 1, wherein the FOV comprises a volume with voxels arranged in three dimensions.

30. A method according to claim 29, wherein the spatially encoding magnetic field is produced by a magnetic field source comprising one or more elements at least some of which change their positions or orientations or both, for the different projections, and wherein the different projections correspond to different values of two independent degrees of freedom, each degree of freedom being a different combination of a position, an orientation, or both, of one or more of the elements.

31. A method according to claim 30, wherein the spatially encoding magnetic field is produced by a magnetic field source that rotates rigidly around the FOV on two or more axes, and the different projections comprise different rotation angles of the magnetic field source around the two or more axes with respect to the FOV.

32. A method according to claim 30, wherein the spatially encoding magnetic field is produced by a magnetic field source that rotates rigidly around the FOV on one axis and translates along the axis, and the different projections comprise different combinations of rotation angle and translation distance of the magnetic field source, with respect to the FOV.

33. A method according to claim 29, also comprising, for each of the projections of the spatially encoding magnetic field:
a) exciting nuclear spins in the FOV with an MRI excitation pulse; and
b) receiving the MRI signal from the excited nuclear spins over a time interval after the excitation pulse;
wherein the spatially encoding magnetic field is produced by a magnetic field source that rotates rigidly around the FOV on one axis, the different projections comprise different rotation angles of the magnetic field source around the axis with respect to the FOV, and components of the signal coming from different distances along the axis are distinguished by the excitation pulse at each projection having a phase that depends nonlinearly on distance along the axis.

34. A method according to claim 1, wherein filtering the received signal comprises using software to calculate the components of the filtered received signal vector from stored data of a received unfiltered signal.

35. A method according to claim 1, wherein filtering the received signal comprises receiving different frequency components of the received signal using different receivers, and recording data of each frequency component only during the time window for that frequency component.

36. A method according to claim 1, wherein for at least one projection, the spatially encoding magnetic field has field gradient magnitudes at different locations within the FOV that differ by at least a factor of 1.5.

37. A method according to claim 36, wherein the standard deviations of the time windows for two or more different frequency component and receiver antenna combinations of the filtered signal, that together contribute at least 10% to a total energy of the filtered signal for that projection, have a standard deviation that is at least 20% of their mean value.

38. A method according to claim 1, wherein the spatially encoding magnetic field has a maximum value in the FOV that is 0.5 tesla or less.

39. A method according to claim 38, wherein the spatially encoding magnetic field has a maximum value in the FOV that is 0.3 tesla or less.

40. A magnetic resonance imaging device for reconstructing an image of a FOV, comprising:
a) a magnetic field source that produces a spatially encoding magnetic field in the FOV, that has a different dependence on position in the FOV at each of a plurality of different projections;
b) one or more MRI excitation antennas that produce an electromagnetic field that resonantly excites nuclear spins in the FOV;
c) a plurality of MRI receiver antennas that receive MRI signals from the excited nuclear spins in the FOV, and one or more MRI receivers that record data of the received MRI signals separately for each projection for at least some different receiver antennas or sets of receiver antennas;
d) a signal filtering module that, for at least some of the projections, filters the received signal by applying time windows of different widths to at least some different components of the signal in different frequency bands, from different receiver antennas, or both, resulting in a filtered received signal vector whose components describe the filtered received signal as a function of time or frequency, at each projection, for each receiver antenna or set of receiver antennas for which the data is recorded separately; and
e) an image reconstruction module configured to find a reconstructed image vector whose components describe a weighted or unweighted net magnetization at each voxel in the FOV, that would be expected to produce the filtered received signal vector.

41. A device according to claim 40, wherein the one or more MRI receivers record data of the unfiltered received MRI signal, and the signal filtering module is configured to calculate the filtered received signal vector from the data of the unfiltered received MRI signal.

42. A device according to claim 40, wherein at least some of the one or more MRI receivers are configured to separately record data of the received MRI signal at each of a plurality of frequency components, and the signal filtering module is configured to limit the recording of data for each frequency component to the time window for that frequency component.

43. A device according to claim 40, comprising a magnetic field source rotator configured to rigidly rotate the magnet field source to a plurality of different orientations around one or more axes relative to the FOV, maintaining a same configuration of the spatially encoding magnetic field relative to the magnetic field source for each of the orientations, the different projections corresponding to the different orientations of the magnetic field source.

44. A device according to claim 40, wherein the magnetic field source comprises an open magnet.

45. A device according to claim 40, wherein the magnetic field source comprises a plurality of magnets, and at least some of the receiver antennas are each located much closer to a different one of the magnets than to the other magnets.

46. A device according to claim 40, wherein the magnetic field source comprises one or more permanent magnets.

47. A device according to claim 46, wherein the one or more permanent magnets comprise a cap-shaped magnet configuration.

48. A device according to claim 40, wherein the magnetic field source comprises at least two elements, the device also comprising a field source element motion system configured to move or rotate one of the elements relative to the other element such that the motion or rotation distorts the spatially encoding magnetic field produced by the magnetic field source.

49. A method of scanning an object in a field of view (FOV) by acquiring an MRI signal from the object in the FOV at a plurality of different projections of a spatially encoding magnetic field generated by a magnetic field source comprising a plurality of magnetic elements at least some of them moveable, and reconstructing an MRI image of the object in the FOV, the method comprising:
a) at each of the projections, acquiring the MRI signal from the FOV;
b) after at least some of the projections, using the magnetic field source to change the spatially encoding magnetic field for the next projection, including, for at least one of those projections, moving at least one of the magnetic elements relative to another of the magnetic elements such that the spatially encoding magnetic field of the next projection is a distortion of the spatially encoding magnetic field of the at least one projection; and
c) reconstructing the MRI image of the object in the FOV from the acquired MRI signal.

50. A method of scanning an object in a FOV according to claim 1, wherein reconstructing the MRI image of the object in the FOV comprises using an encoding matrix that transforms a given image vector of the FOV to a filtered or unfiltered MRI signal vector that the image vector would be expected to produce, and the method also comprises, before acquiring the MRI signal for any of the projections, calibrating the spatially encoding magnetic field at each projection by:
a) placing in the FOV a phantom that produces a known image;
b) acquiring an MRI signal from the FOV for each of the projections;
c) reconstructing an image of the phantom from the MRI signal acquired at each projection;
d) comparing the reconstructed image of the phantom with the known image of the phantom;
e) adjusting one or more components of the encoding matrix, based on differences between the reconstructed image of the phantom and the known image of the phantom, to values that would make the reconstructed image of the phantom more closely match the known image of the phantom; and
f) replacing the phantom in the FOV with the object to be scanned.

51. A method according to claim 36, wherein for at least one projection, the spatially encoding magnetic field is nonlinear, and has a field gradient, for which field gradient magnitudes at different locations within the FOV differ by at least a factor of 1.5.

52. A method according to claim 1, wherein the spatially encoding magnetic field is produced by a magnetic field source comprising one or more coils, and for at least some different projections, a different current is used in one or more of the coils.
